(19) United States
(12) Patent Application Publication
(10) Pub. No.: US 2011/0275635 A1
(43) Pub. Date: Nov. 10, 2011

(54) SMALL MOLECULE INHIBITORS OF NADS, NAMNAT, AND NMNAT

(75) Inventors: Wayne J. Brouillette, Pelham, AL (US); Christie G. Brouillette, Pelham, AL (US); Whitney B. Moro, Birmingham, AL (US)

(73) Assignee: THE UAB RESEARCH FOUNDATION, Birmingham, AL (US)

(21) Appl. No.: 13/143,868

(22) PCT Filed: Jan. 8, 2010
(86) PCT No.: PCT/US2010/020543

§ 371 Date: Jul. 8, 2011

Related U.S. Application Data
(60) Provisional application No. 61/143,637, filed on Jan. 9, 2009.
(60) Provisional application No. 61/166,142, filed on Apr. 2, 2009.

(51) Int. Cl. A61K 31/5377; C07C 311/47; A61K 31/18; C07D 239/69; A61K 31/505; A61K 31/277; A61K 31/196; C07D 413/04; C07D 307/54; A61K 31/341; C07C 311/46; C07C 311/21; A61P 35/00; A61P 31/00; A61P 31/04; C12N 9/99

(52) U.S. Cl. 514/236.2; 435/184; 564/49; 514/597; 544/297; 514/275; 558/413; 514/522; 562/430; 514/562; 544/113; 564/36; 514/590; 549/494; 514/471; 564/86; 514/603; 564/92; 514/604

(57) ABSTRACT

Small molecule inhibitors of bacterial nicotinamide adenine dinucleotide synthetase (NADs), bacterial nicotinic acid mononucleotide adenylyltransferase (NaMNAT), and human nicotinamide mononucleotide adenylyltransferase (NMNAT) are provided, as well as methods of making and using the inhibitors.

### CROSS-REFERENCE TO PRIORITY APPLICATIONS

This application claims priority to U.S. Provisional Application No. 61/143,637, filed Jan. 9, 2009, and U.S. Provisional Application No. 61/166,142, filed Apr. 2, 2009, which are incorporated herein by reference in their entireties.

### STATEMENT REGARDING FEDERALLY FUNDED RESEARCH

This invention was made with government support from the National Institutes of Health Grant numbers U01-AI056477 and U01-AI070386. The government has certain rights in this invention.

### BACKGROUND

Anthrax has been researched as a biological weapon since the early 1920s, and is currently classified by the CDC as a Category A bioterrorism agent. Anthrax poisoning is caused by the rod-shaped, spore-forming bacteria Bacillus anthracis. Bacillus anthracis spores are dormant, and the conversion to the vegetative cell is required for replication and toxin production. The cofactor nicotinamide adenine dinucleotide (NAD) is required for both spore outgrowth and for vegetative growth. Thus, the final two enzymes in the biosynthesis of NAD, bacterial nicotinic acid mononucleotide adenylyltransferase (NaMNAT) and bacterial NAD synthetase (NADs), serve as important targets for treating these and other microbial infections.

### SUMMARY

Compounds and compositions for use as inhibitors of bacterial NAD synthetase (NADs), bacterial nicotinic acid mononucleotide adenylyltransferase (NaMNAT), and/or human nicotinamide mononucleotide adenylyltransferase (NMNAT) are provided herein. A first class of compounds includes compounds of the following formula:

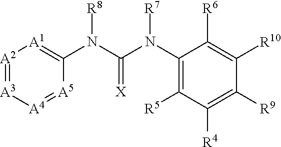

and pharmaceutically acceptable salts thereof. In these compounds, A¹, A², A³, A⁴, and A⁵ are each independently selected from N or CR¹; R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; R⁹ and R¹⁰ are each independently selected from hydrogen and

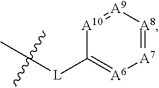

wherein A⁶, A⁷, A⁸, A⁹, and A¹⁰ are each independently selected from N or CR²; and L is —SO₂NR³— or —NR³SO₂—, wherein R⁹ and R¹⁰ are not simultaneously hydrogen; and X is O or S. In this class of compounds, if A¹, A², A⁴, A⁵, A⁶, and A¹⁰ are each CH, A³ is C—NO₂, R⁴, R⁵, R⁶, R⁷, R⁸, and R¹⁰ are hydrogen, X is O, L is SO₂NH, A⁷ is C—Cl, and A⁹ is hydrogen, then A⁸ is not C—Cl. Also, if A¹, A², A⁵, A⁷, A⁸, and A⁹ are each CH, A³ and A⁴ are C—Cl, R⁴, R⁵, R⁶, R⁷, R⁸, and R¹⁰ are hydrogen, X is O, and L is SO₂NH, then A⁶ and A¹⁰ are not simultaneously N. Additionally, if A¹, A⁴, A⁵, A⁶, A⁷, A⁹, and A¹⁰ are each CH, A² and A³ are C—Cl, R⁴, R⁵, R⁶, R⁷, R⁸, and R¹⁰ are hydrogen, X is O, and L is NHSO₂, then A⁸ is not C—CH₃. Further, if A¹, A³, A⁴, A⁵, A⁶, A⁸, and A¹⁰ are each CH, R⁴, R⁵, R⁶, R⁷, R⁸, and R¹⁰ are hydrogen, X is O, L is SO₂NH, A⁷ is C—CF₃, and A⁹ is hydrogen, then A² is not C—Cl or CH.

A second class of compounds includes compounds of the following formula:

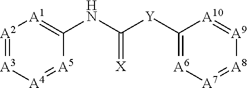

and pharmaceutically acceptable salts or prodrugs thereof. In this class of compounds, A¹, A², A³, A⁴, and A⁵ are each independently selected from N or CR¹; A⁶, A⁷, A⁸, A⁹, and A¹⁰ are each independently selected from N or CR², R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; X is O or S; and Y is —NH—NH—, —NH—CH₂—, an alkyl sulfide, or a sulfonamide. In this class of compounds, if A¹ C—OH, A⁵ is CH, A² and A⁴ are CH, A³ is NO₂, A⁶, A⁸, and A¹⁰ are N, X is O, Y is —CH₂—S—, and A⁹ is aniline, then A⁷ is not

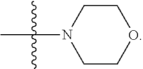

A third class of compounds includes compounds of the following formula:

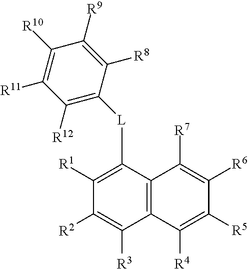

and pharmaceutically acceptable salts or prodrugs thereof. In this class of compounds, L is —SO₂NH— or —NHSO₂—; and R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl. In this class of compounds, if R¹ is nitro, R², R³, R⁴, R⁵, R⁶, R⁷, R⁸, R⁹, R¹¹, and R¹² are hydrogen, and L is SO₂NH, then R¹⁰ is not ethyl.

Also provided herein are compositions including a compound as described above and a pharmaceutically acceptable carrier.

Further provided herein are methods of treating or preventing microbial infections in a subject. A first method of treating or preventing a microbial infection in a subject includes administering to the subject an effective amount a compound of the following structure:

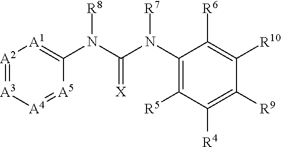

and pharmaceutically acceptable salts and prodrugs thereof, or a composition comprising the compound and a pharmaceutically acceptable carrier. In these methods, A¹, A², A³, A⁴, and A⁵ are each independently selected from N or CR¹; R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; R⁹ and R¹⁰ are each independently selected from hydrogen and

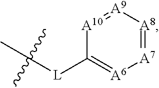

wherein A⁶, A⁷, A⁸, A⁹, and A¹⁰ are each independently selected from N or CR²; and L is —SO₂NR³— or —NR³SO₂—, wherein R⁹ and R¹⁰ are not simultaneously hydrogen; and X is O or S.

A second method of treating or preventing a microbial infection in a subject includes administering to the subject an effective amount a compound of the following structure:

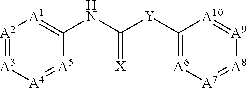

and pharmaceutically acceptable salts or prodrugs thereof, or a composition comprising the compound and a pharmaceutically acceptable carrier. In these methods, A¹, A², A³, A⁴, and A⁵ are each independently selected from N or CR¹; A⁶, A⁷, A⁸, A⁹, and A¹⁰ are each independently selected from N or CR², R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; X is O or S; and Y is —NH—NH—, —NH—CH₂—, an alkyl sulfide, an alkyl carbonyl, or a sulfonamide.

A third method of treating or preventing a microbial infection in a subject includes administering to the subject an effective amount a compound of the following structure:

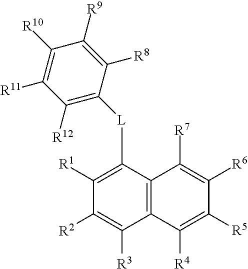

and pharmaceutically acceptable salts or prodrugs thereof, or a composition comprising the compound and a pharmaceutically acceptable carrier. In these methods, L is —SO₂NH— or —NHSO₂—; and R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl.

Methods of making the compounds of the following formula are also described herein:

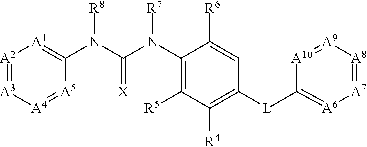

A first method of making the compounds wherein X is O, A¹ is CR¹, A² is CR¹, A³ is CR¹, A⁴ is CR¹, A⁵ is CR¹, A⁶ is CR², A⁷ is CR², A⁸ is CR², A⁹ is CR², A¹⁰ is CR², and one or more of R¹ is NO₂ includes the steps of coupling p-phenylenediamine to a nitrophenylisocyanate to form a 1-(4-aminophenyl)-3-(nitrophenyl)urea and treating the 1-(4-aminophenyl)-3-(nitrophenyl)urea with a benzenesulfonylchloride. A method of making the compounds of the first formula wherein X is S, A¹ is CR¹, A² is CR¹, A³ is CR¹, A⁴ is CR¹, A⁵ is CR¹, A⁶ is CR², A⁷ is CR², A⁸ is CR², A⁹ is CR², A¹⁰ is CR², and one or more of R¹ is NO₂ includes the steps of coupling p-phenylenediamine to a nitrophenylisothiocyanate to form a 1-(4-aminophenyl)-3-(nitrophenyl)thiourea and treating the 1-(4-aminophenyl)-3-(nitrophenyl)thiourea with a benzenesulfonylchloride.

For each of the methods of making described herein, the method can further comprise treating the compound, wherein one or more of R² is cyano, with a reducing agent to form a compound wherein one or more of R² is methylamino. In some examples, the reducing agent is a borane reducing agent.

Also, the methods of making as described herein can further comprise hydrolyzing the compound, wherein one or more of R² is acetamido, to form a compound wherein one or more of R² is amino. In some examples, the hydrolysis is performed using hydrochloric acid in methanol.

Methods of treating or preventing cancer in a subject are further provided herein. A first method of treating or preventing cancer in a subject includes administering to the subject an effective amount a compound of the following structure:

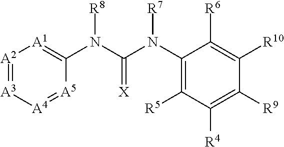

and pharmaceutically acceptable salts and prodrugs thereof, or a composition comprising the compound and a pharmaceutically acceptable carrier. In these to methods, A¹, A², A³, A⁴, and A⁵ are each independently selected from N or CR¹; R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; R⁹ and R¹⁰ are each independently selected from hydrogen and

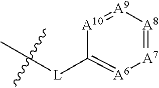

wherein A⁶, A⁷, A⁸, A⁹, and A¹⁰ are each independently selected from N or CR²; and L is —SO₂NR³— or —NR³SO₂—, wherein R⁹ and R¹⁰ are not simultaneously hydrogen; and X is O or S.

A second method of treating or preventing cancer in a subject includes administering to the subject an effective amount a compound of the following structure:

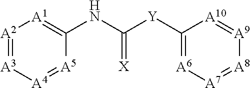

and pharmaceutically acceptable salts or prodrugs thereof, or a composition comprising the compound and a pharmaceutically acceptable carrier. In these methods, A¹, A², A³, A⁴, and A⁵ are each independently selected from N or CR¹; A⁶, A⁷, A⁸, A⁹, and A¹⁰ are each independently selected from N or CR², R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; X is O or S; and Y is —NH—NH—, —NH—CH₂—, an alkyl sulfide, an alkyl carbonyl, or a sulfonamide.

A third method of treating or preventing cancer in a subject includes administering to the subject an effective amount a compound of the following structure:

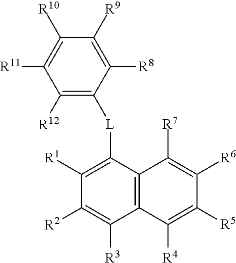

and pharmaceutically acceptable salts or prodrugs thereof, or a composition comprising the compound and a pharmaceutically acceptable carrier. In these methods, L is —SO₂NH— or —NHSO₂—; and R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl.

In some examples, the cancer is breast cancer. The method can further include administering a second compound or composition, wherein the second compound or composition includes an anti-cancer agent.

Methods of inhibiting a bacterial nicotinic acid mononucleotide adenylyltransferase (NaMNAT), bacterial NAD synthetase, bacterial NaMNAT and bacterial synthetase, and human nicotinamide mononucleotide adenylyltransferase (NMNAT) are also provided herein. The methods include contacting the bacterial NaMNAT, bacterial NAD synthetase, bacterial NaMNAT and bacterial synthetase, or human nicotinamide mononucleotide adenylyltransferase (NMNAT) with an effective amount of one or more of the compounds of the following structure:

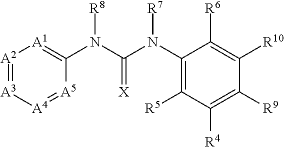

and pharmaceutically acceptable salts and prodrugs thereof, or a composition comprising the compound and a pharmaceutically acceptable carrier. In these methods, A¹, A², A³, A⁴, and A⁵ are each independently selected from N or CR¹; R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; R⁹ and R¹⁰ are each independently selected from hydrogen and

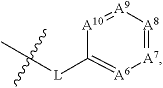

wherein A⁶, A⁷, A⁸, A⁹, and A¹⁰ are each independently selected from N or CR²; and L is —SO₂NR³— or —NR³SO₂—, wherein R⁹ and R¹⁰ are not simultaneously hydrogen; and X is O or S.

A second method of inhibiting a bacterial nicotinic acid mononucleotide adenylyltransferase (NaMNAT), bacterial NAD synthetase, bacterial NaMNAT and bacterial synthetase, or human nicotinamide mononucleotide adenylyltransferase (NMNAT) includes contacting the bacterial NaMNAT, bacterial NAD synthetase, bacterial NaMNAT and bacterial synthetase, or human nicotinamide mononucleotide adenylyltransferase (NMNAT) with an effective amount of one or more of the compounds of the following structure:

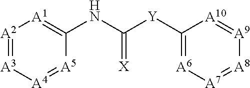

and pharmaceutically acceptable salts or prodrugs thereof, or a composition comprising the compound and a pharmaceutically acceptable carrier. In these methods, A¹, A², A³, A⁴, and A⁵ are each independently selected from N or CR¹; A⁶, A⁷, A⁸, A⁹, and A¹⁰ are each independently selected from N or CR², R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl; X is O or S; and Y is —NH—NH—, —NH—CH₂—, an alkyl sulfide, an alkyl carbonyl, or a sulfonamide.

A third method of inhibiting a bacterial nicotinic acid mononucleotide adenylyltransferase (NaMNAT), bacterial NAD synthetase, bacterial NaMNAT and bacterial synthetase, or human nicotinamide mononucleotide adenylyltransferase (NMNAT) includes contacting the bacterial NaMNAT, bacterial NAD synthetase, bacterial NaMNAT and bacterial synthetase, or human nicotinamide mononucleotide adenylyltransferase (NMNAT) with an effective amount of one or more of the compounds of the following structure:

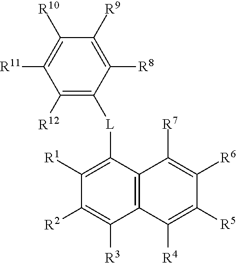

and pharmaceutically acceptable salts or prodrugs thereof, or a composition comprising the compound and a pharmaceutically acceptable carrier. In these methods, L is —SO₂NH— or —NHSO₂—; and R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl.

In some examples of the methods, the contacting occurs in vivo. In some examples of the methods, the contacting occurs in vitro.

Unless otherwise defined, all technical and scientific terms used herein have the same meaning as commonly understood by one of ordinary skill in the art. Although methods and materials similar or equivalent to those described herein can be used in the practice or testing of the described embodiments, suitable methods and materials are described below. All publications, patent applications, patents, and other references mentioned herein are incorporated by reference in their entirety. In case of conflict, the present specification, including definitions, will control. As used herein, the singular forms “a,” “an,” and “the” include plural referents unless the context clearly dictates otherwise. In addition, the materials, methods, and examples are illustrative only and not intended to be limiting.

The details of one or more embodiments are set forth in the description below. Other features, objects, and advantages will be apparent from the description and from the claims.

### DETAILED DESCRIPTION

Bacterial nicotinic acid mononucleotide adenylyltransferase (NaMNAT) and bacterial NAD synthetase are the final two enzymes in the biosynthesis of NAD, a cofactor required for both spore outgrowth and vegetative growth of Bacillus anthracis. The inhibition of either of these enzymes provides antibacterial action at two different steps of the life cycle of the bacterium. Small molecules, including small molecules containing the urea-sulfonamide moiety, have been found that are able to effectively inhibit one or both of these enzymes. Accordingly, inhibition of such enzymes with the administration of the small molecules described herein can provide a method to treat subjects with microbial infections (e.g., bacterial infections). Further, these compounds can be used as human nicotinamide mononucleotide adenylyltransferase (NMNAT) inhibitors for the treatment of cancer.

### A. Compounds

The compounds described herein and pharmaceutically acceptable salts thereof are useful in treating microbial infections and cancer and inhibiting bacterial NaMNAT, bacterial NADs, and human NMNAT. Microbial infections include, for example, bacterial and fungal infections. Bacterial infections include infections caused by bacilli, cocci, spirochaetes, and vibrio bacteria. The compounds described herein are particularly useful against bacterial infections caused by Bacillus anthracis.

A first group of inhibitors includes compounds represented by Formula I:

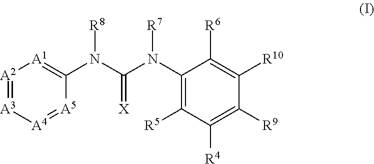

and pharmaceutically acceptable salts and prodrugs thereof.

In Formula I, A¹, A², A³, A⁴, and A⁵ are each independently selected from N or CR¹ and A⁶, A⁷, A⁸, A⁹, and A¹⁰ are each independently selected from N or CR². In some examples, each of A¹, A², A³, A⁴, and A⁵ is CR¹ and each of A⁶, A⁷, A⁸, A⁹, and A¹⁰ is CR².

Also in Formula I, L is —SO₂NR³— or —NR³SO₂—. In some examples, L is NHSO²—.

Additionally in Formula I, R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl. In some examples, one or more of R¹ are each independently selected from hydrogen, nitro, chloro, alkoxyl, or hydroxyl. In some examples, one or more of R² are each independently selected from hydrogen, methyl, ethyl, trifluoromethyl, phenyl, methoxy, phenoxy, amino, methylamino, acetamido, cyano, fluoro, chloro, or carboxyl. In some examples, A⁹ is CR² and R² is selected from methylamino, amino, methoxy, ethyl, or trifluoromethyl. In certain examples, one or more of R² is methylamino. In certain examples, one or more of R² is amino. In certain examples, one or more of R² is methoxy. In certain examples, one or more of R² is ethyl. In certain examples, one or more of R² is trifluoromethyl. In some examples, R⁴, R⁵, and R⁶ are each hydrogen. In some examples, R⁷ and R⁸ are hydrogen.

Also in Formula I, R⁹ and R¹⁰ are each independently selected from hydrogen and

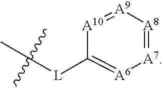

A⁶, A⁷, A⁸, A⁹, and A¹⁰ are each independently selected from N or CR² and L is —SO₂NR³— or —NR³SO₂—

In Formula I, R⁹ and R¹⁰ are not simultaneously hydrogen.

Further in Formula I, X is O or S. In some examples X is O.

In some examples of Formula I, if A¹, A², A⁴, A⁵, A⁶, and A¹⁰ are each CH, A³ is C—NO₂, R⁴, R⁵, R⁶, R⁷, R⁸, and R¹⁰ are hydrogen, X is O, L is SO₂NH, A⁷ is C—Cl, and A⁹ is hydrogen, then A⁸ is not C—Cl.

Also, in some examples of Formula I, if A¹, A², A⁵, A⁷, A⁸, and A⁹ are each CH, A³ and A⁴ are C—Cl, R⁴, R⁵, R⁶, R⁷, R⁸, and R¹⁰ are hydrogen, X is O, and L is SO₂NH, then A⁶ and A¹⁰ are not simultaneously N.

Additionally, in some examples of Formula I, if A¹, A⁴, A⁵, A⁶, A⁷, A⁹, and A¹⁰ are each CH, A² and A³ are C—Cl, R⁴, R⁵, R⁶, R⁷, R⁸, and R¹⁰ are hydrogen, X is O, and L is NHSO₂, then A⁸ is not C—CH₃.

Further, in some examples of Formula I, if A¹, A³, A⁴, A⁵, A⁶, A⁸, and A¹⁰ are each CH, R⁴, R⁵, R⁶, R⁷, R⁸, and R¹⁰ are hydrogen, X is O, L is SO₂NH, A⁷ is C—CF₃, and A⁹ is hydrogen, then A² is not C—Cl or CH.

As used herein, the term “alkyl” includes straight- and branched-chain monovalent substituents. Alkyls useful with the compounds and methods described herein include C₁-C₁₂ alkyls, C₂-C₈ alkyls, and C₃-C₆ alkyls. Examples include methyl, ethyl, isobutyl, and the like. “Heteroalkyl” is similarly defined but may contain O, S, or N heteroatoms or combinations thereof within the backbone. Heteroalkyls useful with the compounds and methods described herein include C₁-C₁₂ heteroalkyls, C₂-C₈ heteroalkyls, and C₃-C₆ heteroalkyls.

The alkyl and heteroalkyl molecules used herein can be substituted or unsubstituted. As used herein, the term “substituted” includes the addition of an organic group to a position attached to the main chain of the alkyl or heteroalkyl, e.g., the replacement of a hydrogen by one of these molecules. Examples of substitution groups include, but are not limited to, hydroxyl, halogen (e.g., F, Br, Cl, or I), and carboxyl groups. Conversely, as used herein, the term “unsubstituted” indicates the alkyl or heteroalkyl has a full complement of hydrogens, i.e., commensurate with its saturation level, with no substitutions, e.g., linear decane (—(CH₂)₉—CH₃).

As used herein, “aryl” refers to aromatic monocyclic or multicyclic groups containing up to 19 carbon atoms. Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. “Heteroaryl” molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline.

Examples of the Formula I include compounds represented by Formula I-A:

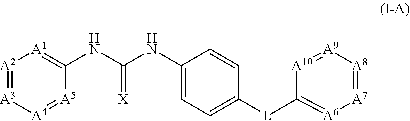

and pharmaceutically acceptable salts and prodrugs thereof.

In Formula I-A, A¹, A², A³, A⁴, and A⁵ are each independently selected from N or CR¹ and A⁶, A⁷, A⁸, A⁹, and A¹⁰ are each independently selected from N or CR².

Also in Formula I-A, R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, or substituted or unsubstituted carboxyl.

Additionally in Formula I-A, L is —SO₂NH— or —NHSO₂—.

Further in Formula I-A, X is O or S.

Additional examples of Formula I include compounds represented by Formula I-B:

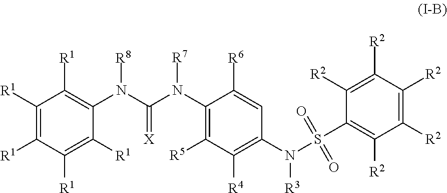

and pharmaceutically acceptable salts thereof.

In Formula I-B, each R¹, each R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl. In some examples, one or more of R¹ are nitro. In some examples, one or more of R² are each independently selected from hydrogen, methyl, ethyl, trifluoromethyl, phenyl, methoxy, phenoxy, amino, methylamino, acetamido, cyano, fluoro, chloro, or carboxyl. In some examples, R² is selected from methylamino, amino, methoxy, ethyl, or trifluoromethyl. In certain examples, one or more of R² is methylamino. In certain examples, one or more of R² is amino. In certain examples, one or more of R² is methoxy. In certain examples, one or more of R² is ethyl. In certain examples, one or more of R² is trifluoromethyl.

Also in Formula I-B, X is O or S. In some examples, X is O.

Examples of the Formula I also include compounds represented by Formula I-C:

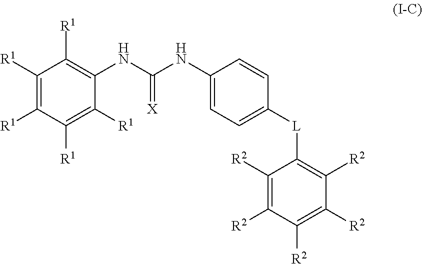

and pharmaceutically acceptable salts and prodrugs thereof.

In Formula I-C, L is —SO₂NH— or —NHSO₂—.

Also in Formula I-C, each R¹ and each R² are independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, or substituted or unsubstituted carboxyl.

Additionally in Formula I-C, X is O or S.

Further examples of inhibitors of Formula I include compounds represented by Formula I-D:

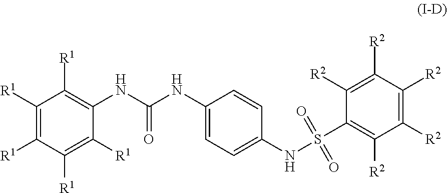

and pharmaceutically acceptable salts thereof.

In Formula I-D, each R¹ and each R² are independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl. In some examples, one or more of R¹ is nitro. In some examples, one or more R² are each independently selected from hydrogen, methyl, ethyl, trifluoromethyl, phenyl, methoxy, phenoxy, amino, methylamino, acetamido, cyano, fluoro, chloro, or carboxyl. In some examples, R² is selected from methylamino, amino, methoxy, ethyl, or trifluoromethyl. In certain examples, one or more of R² is methylamino. In certain examples, one or more of R² is amino. In certain examples, one or more of R² is methoxy. In certain examples, one or more of R² is ethyl. In certain examples, one or more of R² is trifluoromethyl. In some examples, one or more of R¹ is hydrogen.

Particular examples of Formula I include the following compounds:

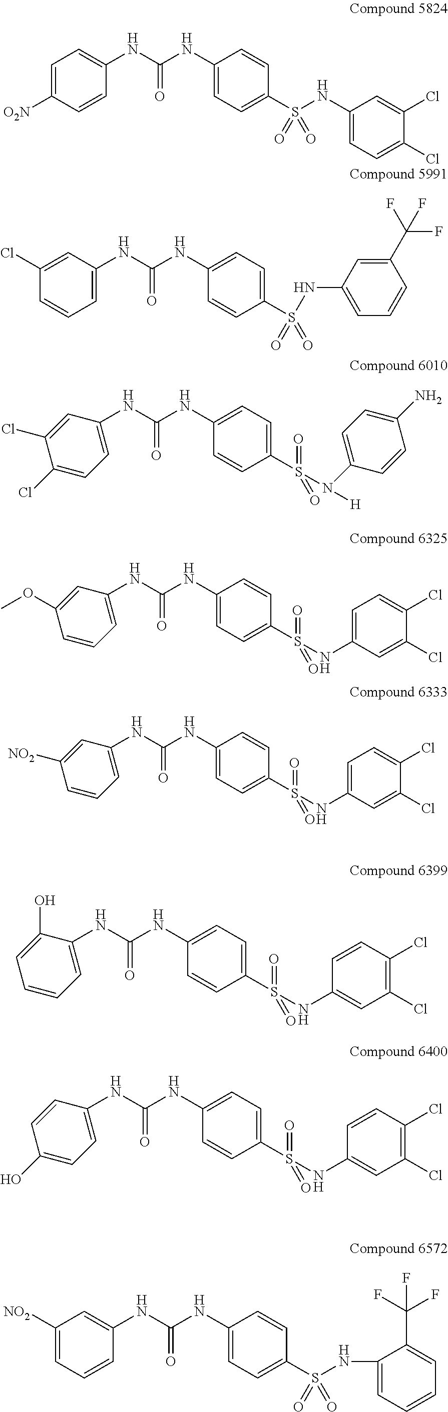

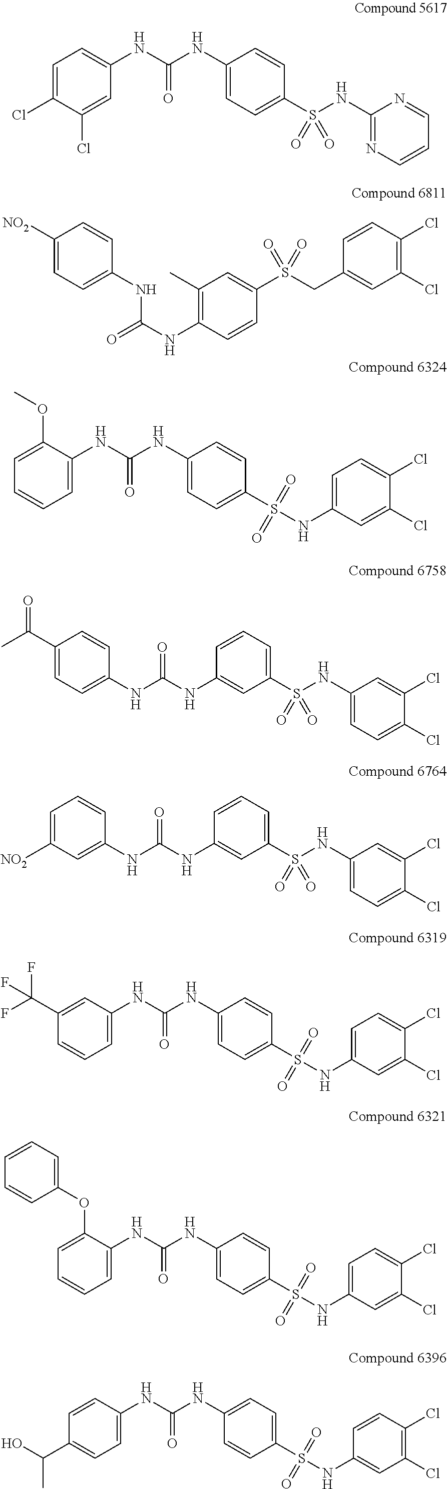

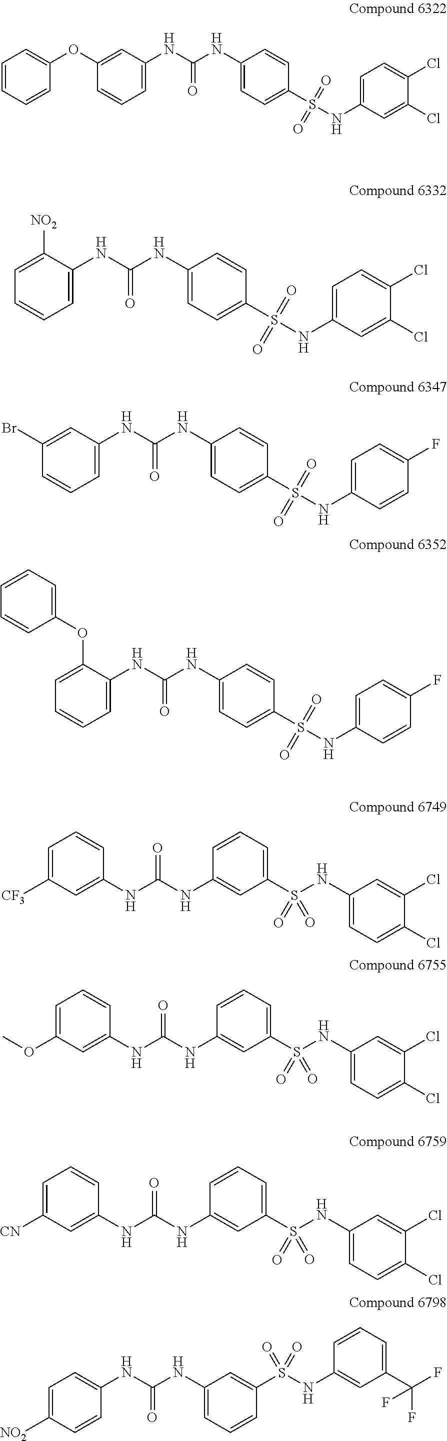

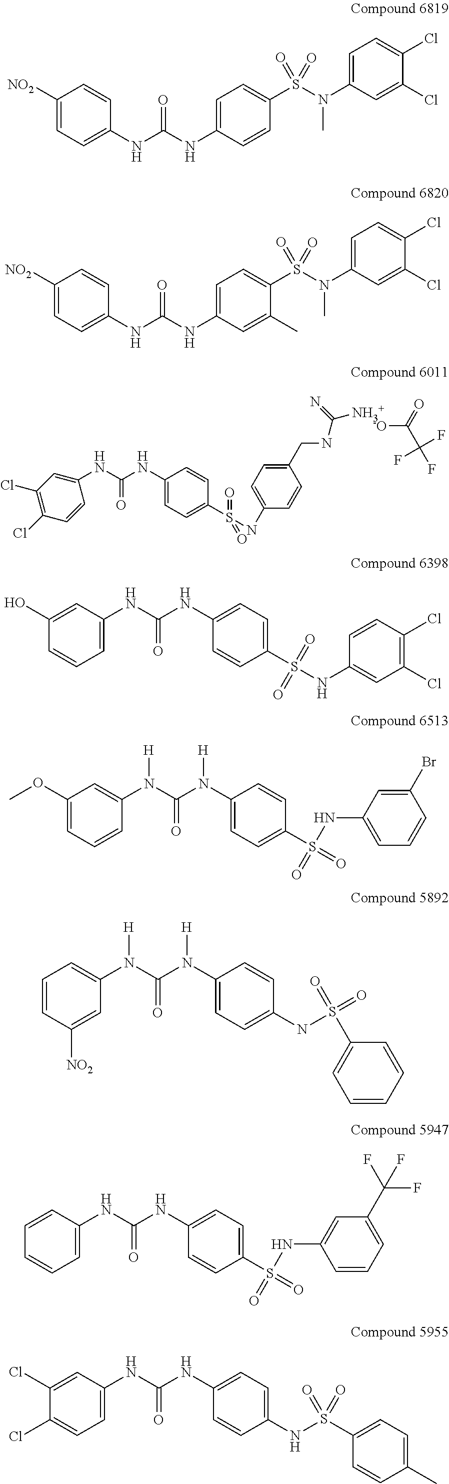

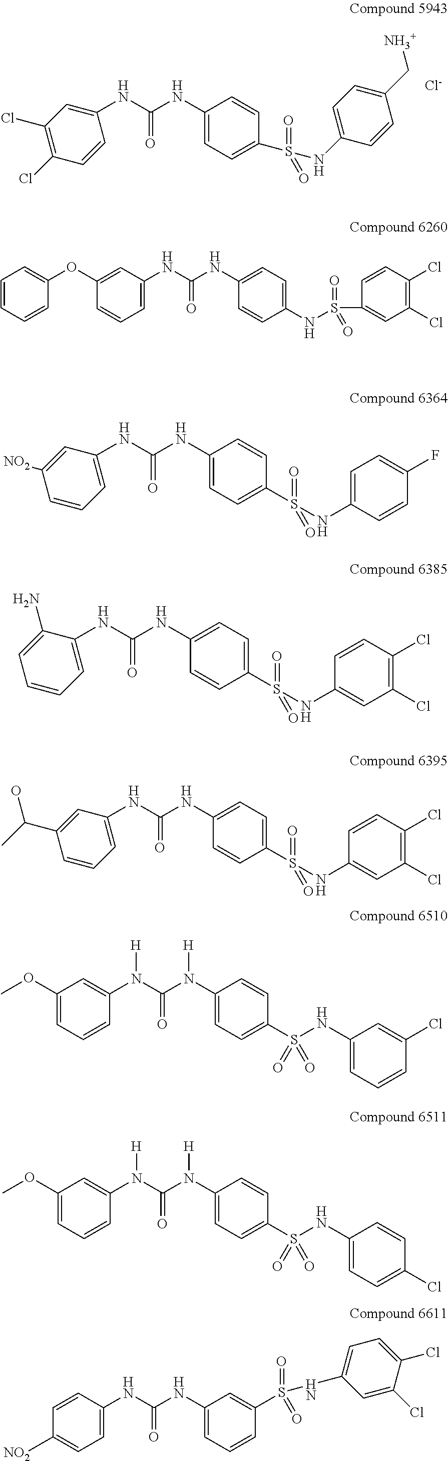

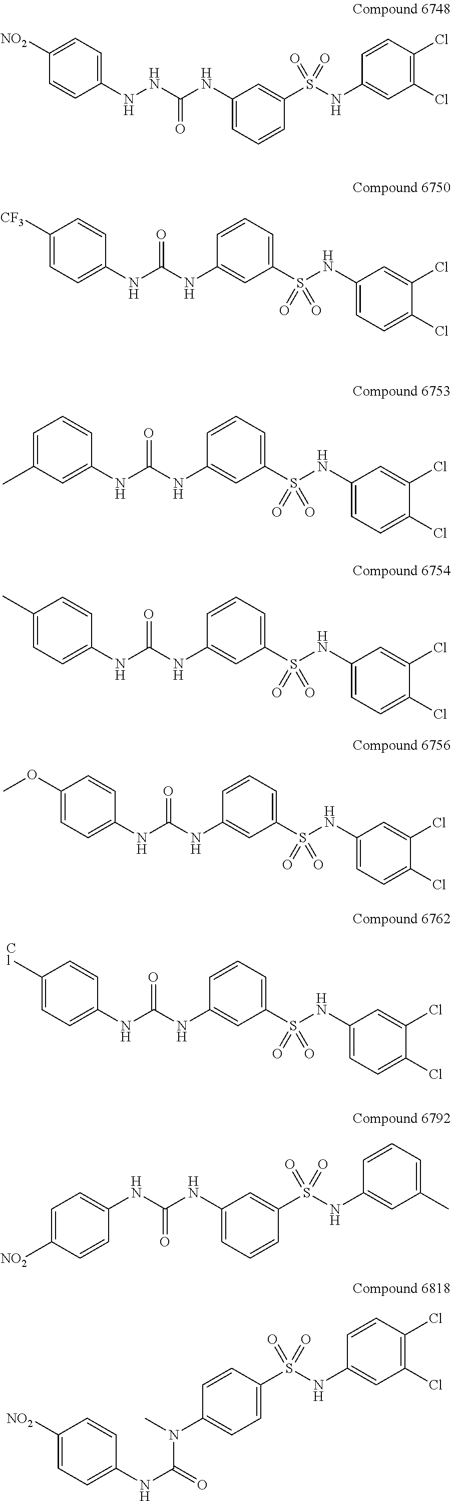

In some examples, Formula I can have the following formula:

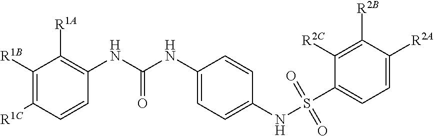

Particular examples are shown in Table 1.

TABLE 1

| R^{1A} | R^{1B} | R^{1C} | R^{2A} | R^{2B} | R^{2C} |
|---|---|---|---|---|---|
| H | H | NO₂ | Cl | Cl | H |
| H | NO₂ | H | Cl | Cl | H |
| NO₂ | H | H | Cl | Cl | H |
| H | H | NO₂ | H | H | Me |
| H | NO₂ | H | H | H | Me |
| NO₂ | H | H | H | H | Me |
| H | H | NO₂ | H | Me | H |
| H | NO₂ | H | H | Me | H |
| NO₂ | H | H | H | Me | H |
| H | H | NO₂ | Et | H | H |
| H | NO₂ | H | Et | H | H |
| NO₂ | H | H | Et | H | H |
| H | H | NO₂ | Ph | H | H |
| H | NO₂ | H | Ph | H | H |
| NO₂ | H | H | Ph | H | H |
| H | H | NO₂ | H | H | F |
| H | NO₂ | H | H | H | F |
| NO₂ | H | H | H | H | F |
| H | H | NO₂ | H | F | H |
| H | NO₂ | H | H | F | H |
| NO₂ | H | H | H | F | H |
| H | H | NO₂ | F | H | H |
| H | NO₂ | H | F | H | H |
| NO₂ | H | H | F | H | H |
| H | H | NO₂ | H | H | Cl |
| H | NO₂ | H | H | H | Cl |
| NO₂ | H | H | H | H | Cl |
| H | H | NO₂ | H | Cl | H |
| H | NO₂ | H | H | Cl | H |
| NO₂ | H | H | H | Cl | H |
| H | H | NO₂ | Cl | H | H |
| H | NO₂ | H | Cl | H | H |
| NO₂ | H | H | Cl | H | H |
| H | H | NO₂ | H | H | CF₃ |
| H | NO₂ | H | H | H | CF₃ |
| NO₂ | H | H | H | H | CF₃ |
| H | H | NO₂ | H | CF₃ | H |
| H | NO₂ | H | H | CF₃ | H |
| NO₂ | H | H | H | CF₃ | H |
| H | H | NO₂ | CF₃ | H | H |
| H | NO₂ | H | CF₃ | H | H |
| NO₂ | H | H | CF₃ | H | H |
| H | H | NO₂ | OPh | H | H |
| H | NO₂ | H | OPh | H | H |
| NO₂ | H | H | OPh | H | H |
| NO₂ | H | H | NHAc | H | H |
| H | H | NO₂ | H | OMe | H |
| H | NO₂ | H | H | OMe | H |
| NO₂ | H | H | H | OMe | H |
| H | H | NO₂ | OMe | H | H |
| H | NO₂ | H | OMe | H | H |
| NO₂ | H | H | OMe | H | H |
| H | H | NO₂ | H | H | CN |
| H | NO₂ | H | H | H | CN |
| NO₂ | H | H | H | H | CN |
| H | H | NO₂ | H | CN | H |
| H | NO₂ | H | H | CN | H |
| NO₂ | H | H | H | CN | H |
| H | H | NO₂ | CN | H | H |
| H | NO₂ | H | CN | H | H |
| NO₂ | H | H | CN | H | H |
| H | H | NO₂ | H | CO₂H | H |
| H | NO₂ | H | H | CO₂H | H |
| NO₂ | H | H | H | CO₂H | H |
| H | H | NO₂ | CO₂H | H | H |
| H | NO₂ | H | CO₂H | H | H |
| NO₂ | H | H | CO₂H | H | H |
| H | NO₂ | H | H | H | CH₂NH₂ |
| NO₂ | H | H | H | H | CH₂NH₂ |
| H | H | NO₂ | H | CH₂NH₂ | H |
| H | NO₂ | H | H | CH₂NH₂ | H |
| NO₂ | H | H | H | CH₂NH₂ | H |
| H | H | NO₂ | CH₂NH₂ | H | H |
| H | NO₂ | H | CH₂NH₂ | H | H |
| NO₂ | H | H | CH₂NH₂ | H | H |
| NO₂ | H | H | NH₂ | H | H |

A second group of inhibitors includes compounds represented by Formula II:

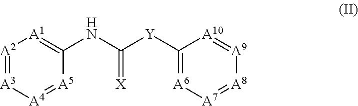

and pharmaceutically acceptable salts and prodrugs thereof.

In Formula II, A¹, A², A³, A⁴, and A⁵ are each independently selected from N or CR¹ and A⁶, A⁷, A⁸, A⁹, and A¹⁰ are each independently selected from N or CR². In some examples, one or more of A⁶, A⁸, or A¹⁰ is N.

Additionally in Formula II, R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, or substituted or unsubstituted carboxyl. In some examples, one or more of A¹, A², A³, A⁴, or A⁵ is CR¹ and R¹ is nitro, chloro, hydroxyl, or alkoxyl. In certain examples, one or more of A⁶, A⁷, A⁸, A⁹, or A¹⁰ is CR² and R² is selected from hydrogen, trifluoromethyl, methoxy, substituted or unsubstituted amino, substituted sulfonamido, chloro, or nitro

Also in Formula II, X is O or S.

Further in Formula II, Y is —NH—NH—, —NH—CH₂—, an alkyl sulfide, an alkyl carbonyl, or a sulfonamide. In some examples of Formula II, Y is not an alkyl carbonyl.

In some examples of Formula II, if A¹ C—OH, A⁵ is CH, A² and A⁴ are CH, A³ is NO₂, A⁶, A⁸, and A¹⁰ are N, X is O, Y is —CH₂—S—, and A⁹ is aniline, then A⁷ is not

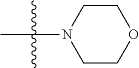

Particular examples of Formula II include the following compounds:

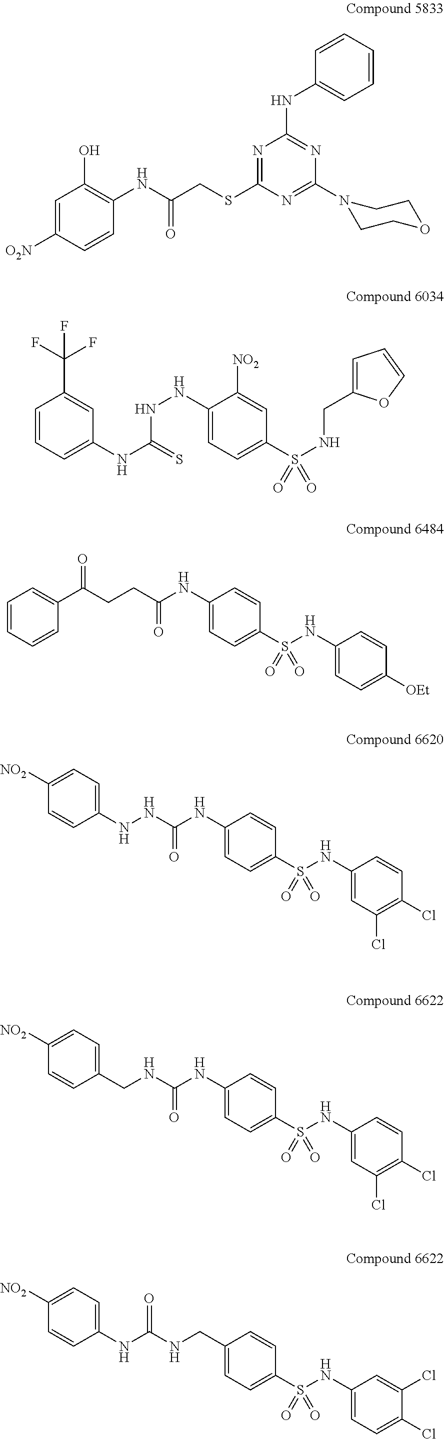

A third group of inhibitors includes compounds represented by Formula III:

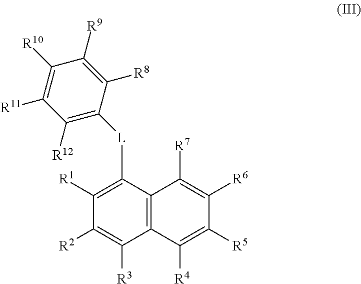

and pharmaceutically acceptable salts and prodrugs thereof.

In Formula III, L is —SO₂NH— or —NHSO₂—.

Also in Formula III, R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, or substituted or unsubstituted carboxyl.

In some examples of Formula III, if R¹ is nitro, R², R³, R⁴, R⁵, R⁶, R⁷, R⁸, R⁹, R¹¹, and R¹² are hydrogen, and L is SO₂NH, then R¹⁰ is not ethyl.

Particular examples of Formula III include the compounds shown below:

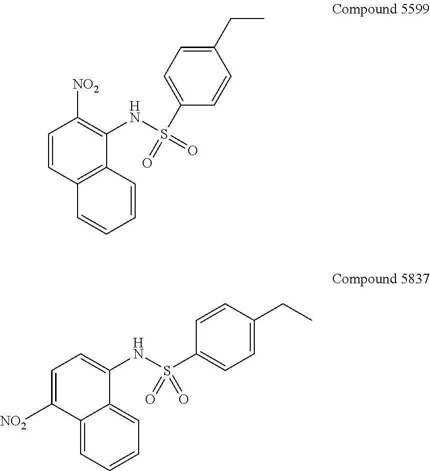

### B. Pharmaceutical Compositions

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught herein.)

Administration of the compounds and compositions described herein or pharmaceutically acceptable salts thereof to a subject can be carried out using therapeutically effective amounts of the compounds and compositions described herein or pharmaceutically acceptable salts thereof as described herein for periods of time effective to treat a disorder. A subject can include both mammals and non-mammals. Mammals include, for example, humans; nonhuman primates, e.g. apes and monkeys; cattle; horses; sheep; rats; mice; pigs; and goats. Non-mammals include, for example, fish and birds.

The effective amount of the compounds and compositions described herein or pharmaceutically acceptable salts thereof as described herein may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. The expression effective amount, when used to describe an amount of compound in a method, refers to the amount of a compound that achieves the desired pharmacological effect or other effect, for example an amount that results in bacterial enzyme inhibition.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

### C. Methods of Making the Compounds

The compounds described herein can be prepared in a variety of ways known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Variations on Formula I, Formula II, and Formula III include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The compounds containing the urea functionality described by Formula I can be made, for example, by coupling p-phenylenediamine to a nitrophenylisocyanate to form a 1-(4-aminophenyl)-3-(nitrophenyl)urea; and treating the 1-(4-aminophenyl)-3-(nitrophenyl)urea with a substituted or unsubstituted benzenesulfonylchloride (see Scheme 1).

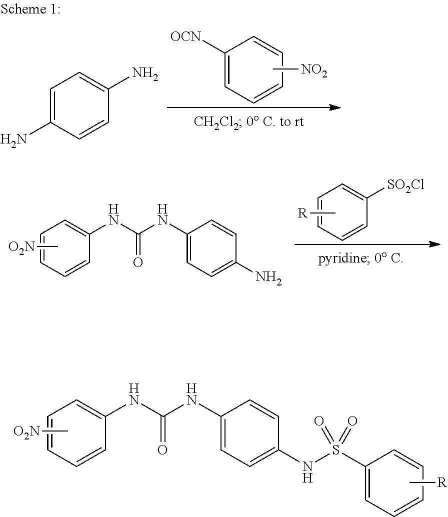

In addition, the compounds containing the thiourea functionality described by Formula I can be made, for example, by coupling p-phenylenediamine to a nitrophenylisothiocyanate to form a 1-(4-aminophenyl)-3-(nitrophenyl)thiourea; and treating the 1-(4-aminophenyl)-3-(nitrophenyl)thiourea with a substituted or unsubstituted benzenesulfonylchloride (see Scheme 2).

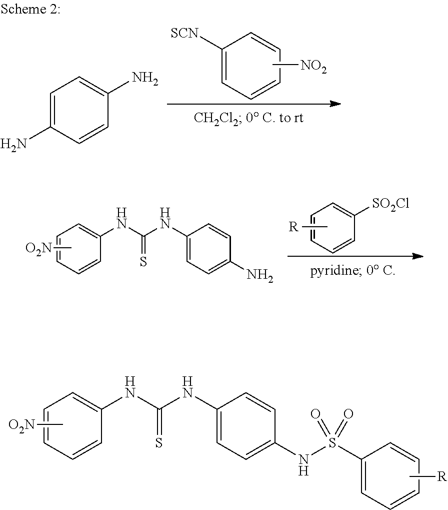

In some examples, the nitrophenylisocyanate is 2-nitrophenyl-isocyanate; 3-nitrophenyl-isocyanate; or 4-nitrophenyl-isocyanate. In some examples, the nitrophenylisothiocyanate is 2-nitrophenyl-isothiocyanate; 3-nitrophenyl-isothiocyanate; or 4-nitrophenyl-isothiocyanate. Also, in some examples, the benzenesulfonyl-chloride is 3,4-dichlorobenzenesulfonylchloride; 2-methylbenzenesulfonylchloride; 3-methylbenzenesulfonylchloride; 4-ethylbenzenesulfonylchloride; 4-phenylbenzene-sulfonylchloride; 2-fluorobenzenesulfonylchloride; 3-fluorobenzenesulfonylchloride; 4-fluorobenzenesulfonylchloride; 2-chlorobenzenesulfonylchloride; 3-chlorobenzene-sulfonylchloride; 4-chlorobenzenesulfonylchloride; 2-trifluoromethylbenzenesulfonyl-chloride; 3-trifluoromethylbenzenesulfonylchloride; 4-trifluoromethylbenzenesulfonyl-chloride; 4-phenoxybenzenesulfonylchloride; 4-acetamidobenzenesulfonylchloride; 3-methoxybenzenesulfonylchloride; 4-methoxybenzenesulfonylchloride; 2-cyanobenzene-sulfonylchloride; 3-cyanobenzenesulfonylchloride; 4-cyanobenzenesulfonylchloride; 3-carboxylbenzenesulfonylchloride; or 4-carboxylbenzenesulfonylchloride. In some examples, the treating step is performed in the presence of a base. In the examples in Scheme 1 and Scheme 2, the base is pyridine.

Certain compounds of Formula I containing a cyano group can be treated with a reducing agent. In these examples, the cyano group is reduced to form a methylamino group, as shown in Scheme 3. In certain examples, the reducing agent is a borane reducing agent, such as a diborane solution (e.g., BH₃.THF), sodium borohydride, and 9-BBN.

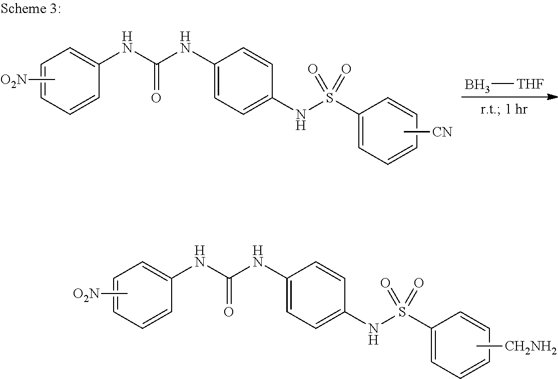

In addition, certain compounds of Formula I containing an acetamido group can be treated with a hydrolyzing agent. In these examples, the acetamido group is hydrolyzed to form an amino group, as shown in Scheme 4. In certain examples, the hydrolysis is performed using hydrochloric acid in methanol.

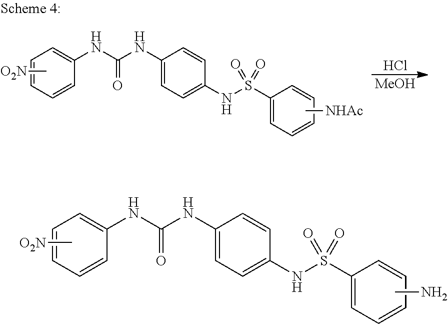

Detailed experimental procedures for synthesizing the compounds described herein can be found in Example 1.

### D. Activity Assays

The activity of the compounds provided herein as inhibitors of bacterial nicotinic acid mononucleotide adenylyltransferase (NaMNAT), bacterial nicotinamide adenine dinucleotide synthetase (NADs), and/or human nicotinamide mononucleotide adenylyltransferase (NMNAT) and may be measured in standard assays, e.g., HPLC assays. Compounds that are identified as NaMNAT inhibitors, NADs inhibitors, or human NMNAT inhibitors are useful in treating or preventing microbial infections and/or cancer. The compounds can be tested as inhibitors of Bacillus anthracis (B. anthracis) NADs in an HPLC assay. The compounds can also be evaluated for antibacterial activity against B. anthracis as described in U.S. Ser. No. 61/143,637, incorporated herein by reference, and Example 1 (below). In some examples, compounds that show activity in the Luria-Bertani (LB) broth antibacterial assay are assayed again using the Mueller Hinton (MH) broth antibacterial assay as specified by the Clinical and Laboratory Standards Institute MIC broth microdilution protocol (see Methods for Dilution Antimicrobial Susceptibility Tests for Bacteria That Grow Aerobically; Approved Standard, In The Clinical and Laboratory Standards Institute (CLSI, formerly NCCLS), 7^{th} ed., January 2006, 26 (2), M7-A7; see also Performance Standards for Antimicrobial Susceptibility Testing; Eighteenth Informational Supplement, In The Clinical and Laboratory Standards Institute (CLSI, formerly NCCLS), January 2008, 28 (1), M100-S18.

Any compound can also be evaluated as an inhibitor of NaMNAT, as described in Examples 1 and 2 (below). The activities of the compounds as determined using the assays described herein can be reported in terms of IC₅₀ and/or MIC 100. As used herein, IC₅₀ refers to an amount, concentration or dosage of a particular test compound that achieves a 50% inhibition of a maximal response in an assay that measures such response. MIC 100 is used to measure the growth inhibition of cells and refers to a 100% inhibition of cell growth.

### E. Methods of Use

Provided herein are methods to treat, prevent, or ameliorate microbial infections and/or cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating microbial infections and cancer in humans, e.g., pediatric and geriatric populations, and in animals, e.g., veterinary applications. Microbial infections include, for example, bacterial and fungal infections. Bacterial infections include infections caused by bacilli, cocci, spirochaetes, and vibrio bacteria. In some examples, the microbial infection is a bacterial infection (e.g., a gram positive bacterial infection). In some examples, the bacterial infection is B. anthracis, B. cereus, E. faecalis, vancomycin resistant E. faecium (i.e., E. faecium VRE), S. aureus, methocillin reistant S. aureus (S. aureus MRSA), or S. pneumoniae. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

Also provided herein are methods of inhibiting bacterial or human NaMNAT and/or bacterial NAD synthetase. The methods comprise contacting the bacterial or human NaMNAT and/or bacterial NAD synthetase with an effective amount of one or more of the compounds or compositions described herein. Such amounts are sufficient to achieve a therapeutically effective concentration of the compound or active component of the composition in vivo or in vitro.

These methods can further include treatment with one or more additional agents (e.g., an antiviral, an antibiotic, or an anti-cancer agent). The one or more additional agents and the compounds and compositions or pharmaceutically acceptable salts thereof as described herein can be administered in any order, including simultaneous administration, as well as temporally spaced order of up to several days apart. The methods may also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein may be by the same or different routes. When treating with one or more additional agents, the compounds and compositions or pharmaceutically acceptable salts thereof as described herein can be combined into a pharmaceutical composition that includes the one or more additional agents. For example, the compounds and compositions or pharmaceutically acceptable salts thereof as described herein can be combined into a pharmaceutical composition with an antibiotic, for example, a penicillin, a cephalosporin, a polymixins, a quinolone, a sulfonamide, an aminoglycoside, a macrolide, a tetracycline, a cyclic lipopeptides, a glycylcycline, and an oxazolidinone. Additionally, the compounds or compositions or pharmaceutically acceptable salts thereof as described herein can be combined into a pharmaceutical composition with an additional anti-cancer agent, such as abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, or zoledronate. The additional anti-cancer agent can also include biopharmaceuticals such as, for example, antibodies.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of a microbial infection or cancer), during early onset (e.g., upon initial signs and symptoms of a microbial infection or cancer), or after an established microbial infection or development of cancer. Prophylactic administration can occur for several days to years prior to the manifestation of symptoms of an infection. Prophylactic administration can be used, for example, in the preventative treatment of subjects exposed to Bacillus anthracis. Therapeutic treatment involves administering to a subject a therapeutically effective amount of the compounds and compositions or pharmaceutically acceptable salts thereof as described herein after a microbial infection or cancer is diagnosed.

### F. Kits

Also provided herein are kits for treating or preventing a microbial infection in a subject. A kit can include any of the compounds or compositions described herein. For example, a kit can include a compound of Formula I, Formula II, Formula III, or combinations thereof. A kit can further include one or more antibacterial agents (e.g., penicillin). A kit can also include one or more anti-cancer agents (e.g., paclitaxel). A kit can include an oral formulation of any of the compounds or compositions described herein. A kit can additionally include directions for use of the kit (e.g., instructions for treating a subject).

### EXAMPLES

### Example 1A

### Virtual Screening to Identify Lead Inhibitors for Bacterial NAD Synthetase (NADs)

The in silico screening program FlexX 1.20.1 (BioSolveIT GMBH; Cologne Area, Germany) was used for the virtual screening of commercially available compounds within the catalytic site of NADs to identify new classes of lead inhibitors. In this study, four commercial compound databases were filtered according to Lipinski's rule of 5 using Tripos' (St. Louis, Mo.) program Unity: Maybridge (58,650 after filtering), ChemBridge (404,132), Tripos' LeadQuest (72,660), and ComGenex (82,737). Because these docking studies predate the solution of the crystal structure of B. anthracis NADs (McDonald et al. Acta Crystallographica, Section D-Biological Crystallography 2007, 63, 891), the highest available resolution crystal structure of B. subtilis NADs (Symersky et al. Acta Crystallographica, Section D-Biological Crystallography 2002, 58 (Part 7), 1138) was utilized for docking. The crystal structures of B. anthracis and B. subtilis NADs reveal that the binding sites are nearly identical, with all active site residues being conserved (McDonald et al. Acta Crystallographica, Section D-Biological Crystallography 2007, 63, 891).

NADs is a large homodimer of approximately 60 kDa that contains two identical binding sites, one within each monomer. The crystal structure (PDB code 1KQP) of the protein from B. subtilis reveals two identical long, linear binding sites containing the adenylated reaction intermediates lying partly within the dimer interface on the NaAD end, and in a buried cavity within one monomer on the ATP end. Due to the enormity of the NADs homodimer catalytic site, and considering the limited computational resources at that time, three smaller binding subsites were constructed to be used in the virtual screening study. To accomplish this, a sphere with radius 25 Å around one of the bound intermediates was extracted from the whole protein structure to produce a partial protein structure which consisted of the three shells of amino acid residues immediately surrounding the binding cavity and which fully contained one complete binding site. All crystallographic waters and metals were removed, hydrogens were added, and the protonation states of active site residues were adjusted to their dominant ionic forms assuming a local physiological pH. The “active site,” as needed for use by FlexX, was further defined by creating a smaller sphere of radius 17 Å which consisted of the first two shells of amino acids surrounding the bound substrate, resulting in a rather large active site: 31 Å in length, and a width ranging from 7 Å on the NaAD end to 16 Å on the ATP end.

As explained earlier, the complete catalytic site was then divided into three overlapping subsites: the NaAD binding subsite, the ATP subsite, and a center subsite which bridges the two end sites. The resulting NaAD binding subsite is the most confined and is approximately 16 Å long and 7 Å wide, appearing as a “canyon” near the homodimer interface; the center subsite is shaped like a tunnel, and is 14 Å long and 9 Å wide; the ATP subsite is buried within a single monomer and is the largest of the three at 21 Å long and 16 Å in width. The bound ligand was excluded from all docking runs.

Each of the four commercial databases was docked into each of the three subsites employing FlexX 1.20.1, which has been shown to be suitable for exploring many kinds of binding sites (Lyne, P. D. et al., J. Med. Chem. 2004, 47, 1962; Stahl, M. and Rarey, M. J. Med. Chem. 2001, 44, 1035; Luksch, T. et al. Chem. Med. Chem. 2008, 3, 1323) and routinely produces hit rates comparable to other highly regarded programs (Kontoyianni et al. J. Comput. Chem. 2005, 26, 11; Bursulaya et al. J. Comput.-Aided Mol. Des. 2003, 17, 755; Rarey et al. Bioinformatics 1999, 15, 243). FlexX was accessed using the SYBYL 6.9 suite of programs (Tripos, Inc.; St. Louis, Mo.), and default parameters were used for each docking run. Automatic base fragment selection was employed. Within each of the three subsites, the core subpocket was defined as all residues which interact directly with the bound substrate. Formal charges were assigned, and 5 poses for each ligand were saved. Docking began on a 64 bit dual processor SGI Octane computer running Unix (Silicon Graphics, Inc; Sunnyvale, Calif.), and was completed in parallel using a 64 bit PQS 4-processor Opteron Quantum Cube running Linux (Advanced Micro Devices, Inc.; Sunnyvale, Calif.). After all databases were screened against all sites and ranked according to FlexX score, the best poses of each docked ligand were re-ranked using a consensus scoring program, CScore (Tripos; St. Louis, Mo.) (Yang et al. J. Chem. Inf. Model. 2005, 45, 1134; Wang, et al. J. Chem. Inf. Comput. Sci. 2001, 41, 1422; Dessalew et al. Biophys. Chem. 2007, 128, 165; Forino et al. J. Med. Chem. 2005, 48, 2278). A total of 22,240 compounds were ranked with CScore, and all compounds with a CScore of 5 were reviewed according to several criteria: realistic orientation within the binding pocket, a predicted binding conformation that is energetically reasonable, structures that are chemically simple and can be easily modified synthetically, and compounds representative of chemically diverse structural classes that are considered medicinally interesting. Additionally, selected compounds with both a CScore of 4 and a good FlexX score were reviewed if they were structurally unique. Representatives from the most interesting structural classes were purchased and screened in NADs enzyme inhibition and B. anthracia antibacterial assays.

The high-throughput assay utilized for previous synthetic NAD synthetase inhibitors (Velu et al. J. Comb. Chem. 2005, 7, 898) monitored production of NAD via enzymatic conversion to NADH, and the latter was detected by both fluorescence and UV absorption. However, this assay was unsuitable for many commercial compounds because they interfered with the fluorescence and/or absorbance at the wavelengths observed. Further, some compounds gave false positives due to direct reaction with NADH. Therefore, an alternate HPLC assay was designed and is presented here for the first time.

In this new assay the reaction product NAD was directly monitored. Sample plates were prepared using a BioMek FX liquid handling system (Beckman Coulter; Brea, Calif.) and the reaction volume was 200 μL. The reaction mixture contained 60 mM HEPPS, pH 8.5, 0.5 mM NH₄Cl, 20 mM KCl, 10 mM MgCl₂, 0.1 mM NaAD, 0.2 mM ATP, 6 μg/ml purified B. anthracis NADS, 2.5% (v/v) DMSO, 0.3% BOG and inhibitors at various concentrations. Compounds were assayed beginning at 600 μM and at doubling dilutions down to 0.6 μM. The reaction was initiated by adding 0.2 mM ATP, and quenched after 10 minutes by adding 50 μL of 6 M guanidine-HCl. The plates were sealed by aluminum tape, and centrifuged at 2500 rpm for 10 minutes in order to pellet any precipitation that may have been caused by the inhibitors. Plates were stored at 4° C. prior to the HPLC analysis.

The HPLC procedure utilized a Gilson 215 liquid handler, two Gilson 306 pumps, and a Gilson 170 diode array detector (Gilson, Inc.; Middleton, Wis.). A Phenomenex Luna 5 μm, C5, 100 Å, 100×4.60 mm column (Phenomenex, Inc.; Torrance, Calif.) was used for separations. The mobile phase was A: 20 mM NaH₂PO₄ pH 6.90 and B: acetonitrile. The gradient was 100% A from 0-3 minutes, to 5% A/95% B from 3-4 minutes for each 20 μL injection. The flow rate was 1.0 mL/min and DAD detection was 190-400 nm Peak height estimation for NAD was based on baseline integration. The % inhibition at each inhibitor concentration was calculated by the difference in peak height of NAD compared to reactions without inhibitor. The IC₅₀ was determined from the plot of NAD peak height vs inhibitor concentration, and is defined as the concentration of inhibitor required to produce NAD peak height at 50% of the uninhibited reaction. Each compound was tested in duplicate, and the IC₅₀ is reported as the average IC₅₀ obtained from duplicate runs. False positives due to promiscuous inhibition were excluded by including detergents in the inhibition assay.

All purchased commercial compounds were also screened against Bacillus anthracis Sterne in an antibacterial assay as previously reported (Tritz, G. J. In Escherichia coli and Salmonella typhimurium Cellular and Molecular Biology. Neidhardt, F. C. Ingraham, J. L., Brooks Low, K., Magasanik, B., Schaechter, M., Umbarger, H. E., Eds., Washington, D.C.: American Society for Microbiology, 1987, Vol. 1, pp 557-563; Velu et al. J. Comb. Chem. 2005, 7, 898) with the following modifications. B. anthracis Sterne spores were subcultured from stock cultures into Luria-Bertani (LB) broth and incubated for 2-3 hours at 37° C. in ambient air until the OD₆₀₀ measurement reached 0.5 to 0.6, when the bacteria were in mid-log phase. The cultures were diluted 1:1 into LB Broth with an absorbance at 600 nm measuring 0.25 to 0.3, then were added to plates containing 240 μM samples of the compounds to be tested. Compounds were tested at a final DMSO concentration of 1%. The plates were incubated at 37° C., and absorbance at 600 nm was read at 0 hour and every hour for 5 hours. Any compounds which inhibited growth of the vegetative cell (as compared to the control containing only DMSO) were screened in a full MIC determination starting at 240 μM and creating doubling dilutions down to 1.88 μM in quadruplicate wells. A plot of cell density vs. time yields inhibition of growth results, and the MIC is defined as the lowest concentration of compound required to completely inhibit growth (100% inhibition). MIC₁₀₀ is reported as the average of the four data points acquired for each compound. Controls for each assay measured sterility, B. anthracia Sterne viability, and included a commercial antibiotic positive control (ciprofloxacin hydrochloride from MP Biomedicals; Solon, Ohio).

Among the NADs subsites, the best FlexX scores were obtained from docking in the larger ATP subsite, presumably due to the many residues capable of charge-charge interactions. A total of 211 commercial compounds were purchased based on the CScore rankings: 135 from the NaAD, 31 from the center and 45 from the ATP subsites; 42 of those compounds were found to have IC₅₀'s less than or equal to 300 μM against NADs (Table 2). Structures of the compounds listed in Table 2 are provided in Table 3.

TABLE 2

| ID | MW | NADs subsite | IC₅₀ (μM) | MIC₁₀₀ (μM) |
|---|---|---|---|---|
| 5379 | 278.27 | NaAD | 51 | 120 |
| 5588 | 466.84 | ATP | 78.5 | >215 |
| 5589 | 378.34 | center | 136.6 | >264 |
| 5591 | 364.32 | center | 160 | >274 |
| 5597 | 446.48 | ATP | 86.1 | >224 |
| 5599 | 356.40 | center | 168.1 | 3.75 |
| 5604 | 450.54 | ATP | 141 | >222 |
| 5605 | 368.37 | ATP | 145.9 | >259 |
| 5606 | 422.37 | center | 141.1 | >237 |
| 5609 | 490.61 | ATP | 70 | >204 |
| 5615 | 449.40 | ATP | 55.4 | >223 |
| 5616 | 404.21 | center | 207.5 | >247 |
| 5617 | 438.29 | center | 77.5 | 15 |
| 5660 | 258.23 | NaAD | 22.5 | >387 |
| 5679 | 303.71 | NaAD | 262 | >329 |
| 5684 | 440.26 | NaAD | 99.5 | >227 |
| 5691 | 430.25 | NaAD | 106 | >232 |
| 5707 | 424.43 | ATP | 253 | >240 |
| 5710 | 327.39 | NaAD | 128.5 | >240 |
| 5724 | 443.44 | NaAD | 290.6 | >240 |
| 5731 | 506.92 | center | 270.7 | >240 |
| 5737 | 354.39 | NaAD | 235.3 | >240 |
| 5749 | 527.76 | NaAD | 219.8 | >240 |
| 5763 | 472.89 | NaAD | 232.1 | >240 |
| 5764 | 505.96 | NaAD | 97.2 | >240 |
| 5768 | 455.50 | center | 170.5 | >240 |
| 5775 | 432.33 | NaAD | 290 | >240 |
| 5785 | 426.39 | center | 108.6 | >240 |
| 5792 | 346.35 | NaAD | 76 | >240 |
| 5793 | 465.52 | NaAD | 78.8 | >240 |
| 5798 | 472.68 | NaAD | 61.8 | >240 |
| 5799 | 479.45 | NaAD | 174.8 | >240 |
| 5802 | 411.42 | NaAD | 225.2 | >240 |
| 5806 | 413.44 | NaAD | 67.8 | >240 |
| 5807 | 401.40 | NaAD | 123.9 | >240 |
| 5815 | 404.47 | NaAD | 185.6 | >240 |
| 5818 | 494.51 | NaAD | 65.7 | >240 |
| 5821 | 411.80 | NaAD | 103.6 | >240 |
| 5822 | 424.46 | NaAD | 107.1 | >240 |
| 5824 | 481.32 | NaAD | 10 | 1.9 |
| 5830 | 441.49 | NaAD | 198.2 | >240 |
| 5831 | 451.89 | NaAD | 243.3 | >240 |
| 5833 | 483.51 | NaAD | 78.3 | 15 |

It should be noted that ranking compounds solely by their FlexX scores produced fewer hits than when compounds were ranked using consensus scoring. At 100 μM or below, 18 compounds (8.5% hit rate) were active against NADs (a cutoff routinely used to define virtual screening hit rates) (Doman et al. J. Med. Chem. 2002, 45, 2213; Perola et al. J. Med. Chem. 2000, 43, 401; Shoichet et al. Curr. Opin. Chem. Biol. 2002, 6, 439), while 6 (2.8% hit rate) were active at or below 50 μM. The hit rate at 100 μM is similar to those obtained by other virtual screening studies against different enzymatic targets (Perola et al. J. Med. Chem. 2000, 43, 401; Shoichet et al. Curr. Opin. Chem. Biol. 2002, 6, 439; Bissantz et al. J. Med. Chem. 2000, 43, 4759). Of these active compounds, 27 inhibitors resulted from their predicted binding in the NaAD subsite, while 9 and 7 were predicted to bind in the center and ATP sites, respectively. The hit rates (100 μM) based on the number of compounds purchased from the NaAD, center, and ATP subsites were 8.9%, 9.7%, and 8.9%, respectively. Only a few compounds scored well in more than one subsite, and none of those screened were enzyme inhibitors.

Drug-like compounds having good activities against both NADs and B. anthracia were identified from this study: 5617, 5824, and 5833. However, unlike earlier tethered dimer inhibitors, there is a poor correlation between enzyme inhibition and antibacterial effects. Several enzymatically inactive commercial compounds were found to behave as antibacterial agents, while only 4 compounds that inhibited NADs were also effective against the vegetative cell, with MIC's at or below 15 μM. This is in contrast to results for earlier libraries of tethered dimer NADs inhibitors, which exhibited a linear correlation between enzyme inhibition and antibacterial activity (Nessi et al. J. Biol. Chem. 1995, 270, 6181). Possible explanations for active enzyme inhibitors that do not show a good MIC include: (1) low permeability into the bacterial cell; (2) loss via efflux pumps (Walsh et al. Chem. Rev. 2005, 105, 391); or (3) metabolism by the bacterial cell into inactive forms. It can also be inferred that those compounds which confer antibacterial activity against the vegetative cell but do not inhibit NADs must be acting on a different target(s).

Among the enzyme inhibitors identified, several different structural classes have emerged (Table 3), and those that also inhibit bacterial growth are considered most interesting for further optimization. 5379 is an acrylonitrile—potentially a good Michael acceptor, and thus may not be an ideal drug candidate. Other structural classes that produced NADs inhibitors include sulfonamides (5599, 5617 and 5824), ureas (5609, 5617, and 5824), complex amides (5615, 5798, 5818 and 5833), and Schiff bases (5660). Except for 5833, all of the antibacterial inhibitors (5599, 5617 and 5824) contain a sulfonamide, a urea, or a combination of both. While all four of these antibacterial inhibitors meet the requirements for moderate molecular weight in a drug-like structure, with the possibility for further analog generation, we selected 5617 and 5824 as compounds that best meet these requirements. 5833 appears less suitable for facile synthetic modifications, and the o-nitronaphthylamine moiety of 5599 contains two lower ranking functionalities relative to drug potential (e.g., the nitro and naphthalene groups). Compounds 5617 and 5824 reveal several similarities; their enzyme and antibacterial activities are very similar, both contain three aryl rings linked by a urea and a sulfonamide, and both contain a 3,4-dichlorophenyl ring.

TABLE 3

| | | NADs | B.a. |
|---|---|---|---|
| Cmpd. | | IC₅₀ | MIC |
| ID | Structure | (μM) | (μM) |
| Cipro | 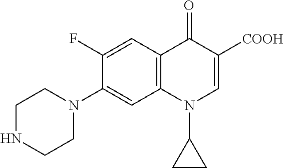 | — | 0.5 |
| 5824 | 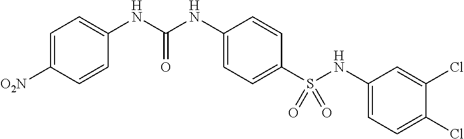 | 10 | 1.9 |
| 5599 | 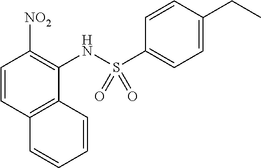 | 168.1 | 3.75 |
| 5617 | 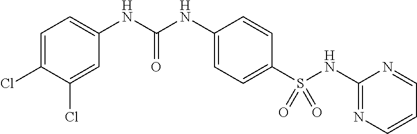 | 77.5 | 15 |
| 5833 | 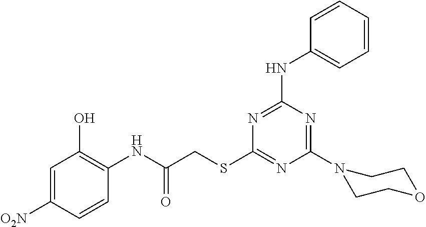 | 78.3 | 15 |
| 5660 | 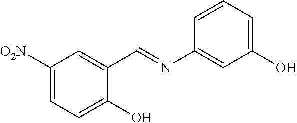 | 22.5 | >387 |
| 5379 | 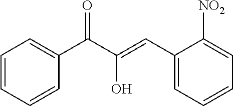 | 51 | 120 |
| 5615 | 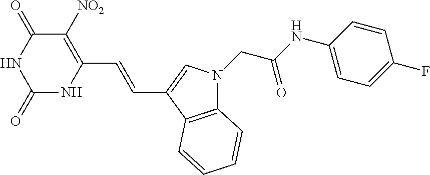 | 55.4 | >223 |
| 5798 | 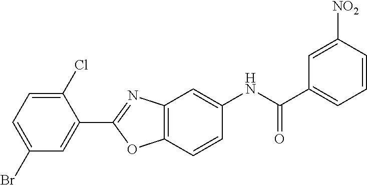 | 61.8 | >240 |
| 5818 | 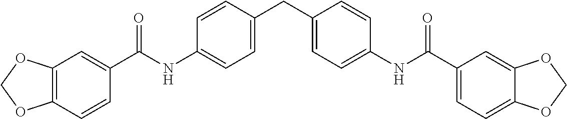 | 65.7 | >240 |
| 5609 | 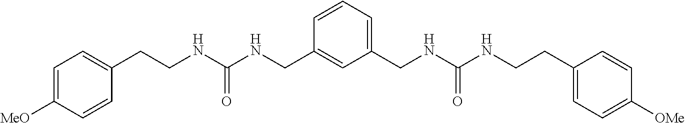 | 70 | >204 |
| 5588 | 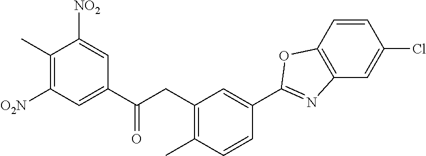 | 84 ± 35 | >240 |
| 5589 | 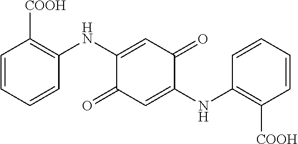 | 118 ± 67 | >264 |
| 5591 | 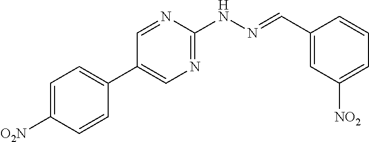 | 160 ± 57 | >274 |
| 5597 | 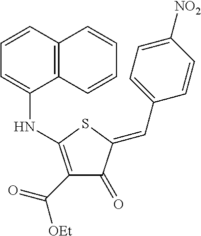 | 86 ± 33 | >224 |
| 5604 | 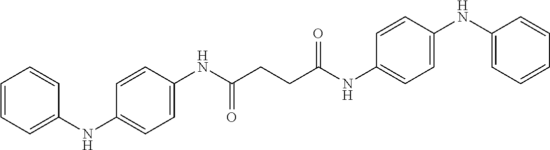 | 256 ± 210 | >222 |
| 5605 | 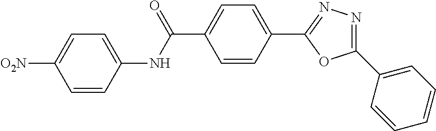 | 146 ± 0.2 | >259 |
| 5606 | 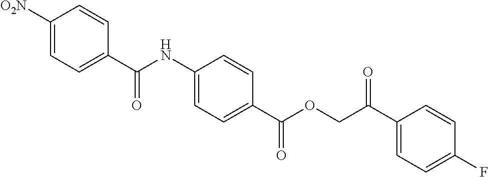 | 176 ± 49 | >237 |
| 5616 | 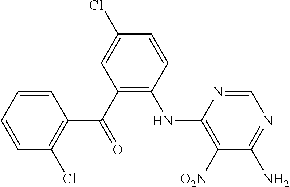 | 231 ± 36 | >247 |
| 5679 | 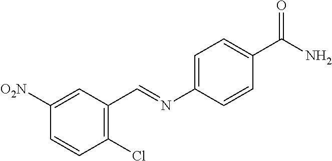 | 262 ± 8 | >329 |
| 5684 | 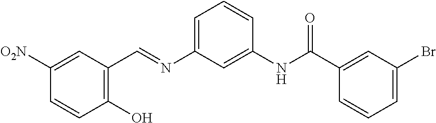 | 99 ± 12 | >227 |
| 5691 | 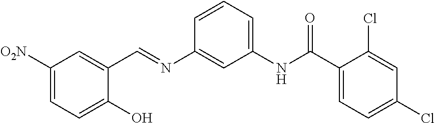 | 106 ± 2 | >232 |
| 5707 | 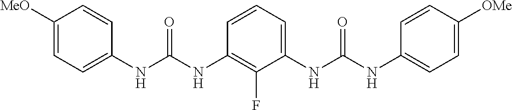 | 253 ± 65 | >240 |
| 5710 | 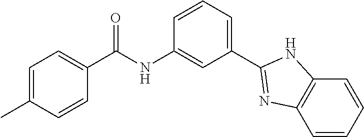 | 189 ± 86 | >240 |
| 5724 | 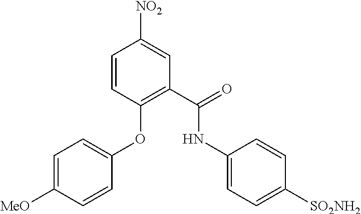 | 291 ± 29 | >240 |
| 5731 | 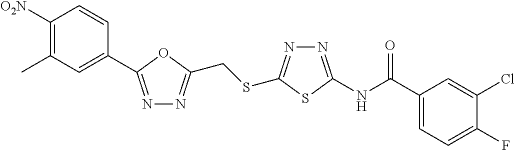 | 270 ± 3 | >240 |
| 5737 | 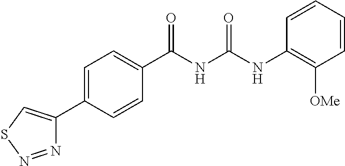 | 235 ± 71 | >240 |
| 5749 | 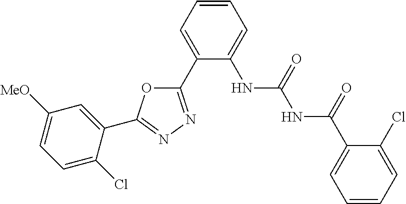 | 220 ± 33 | >240 |
| 5763 | 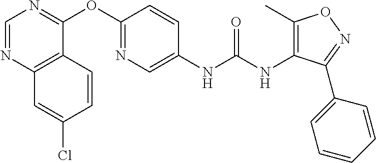 | 232 ± 55 | >240 |
| 5764 | 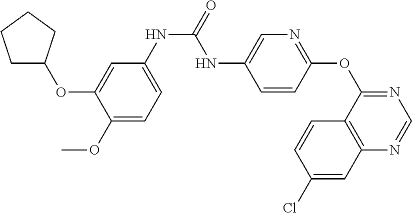 | 97 ± 55 | >240 |
| 5768 | 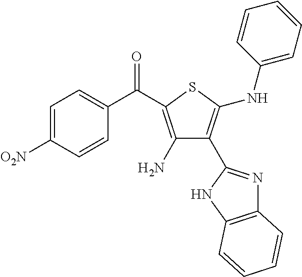 | 170 ± 48 | >240 |
| 5775 | 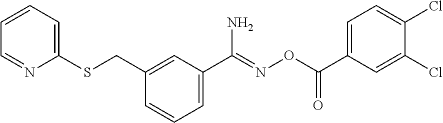 | 290 ± 0 | >240 |
| 5785 | 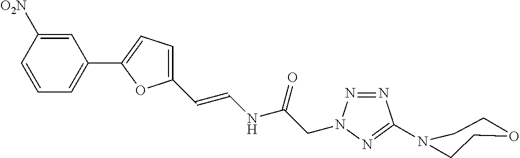 | 109 ± 12 | >240 |
| 5792 | 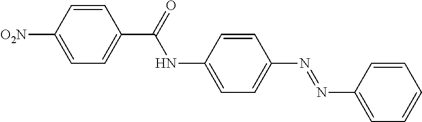 | 76 ± 13 | >240 |
| 5793 | 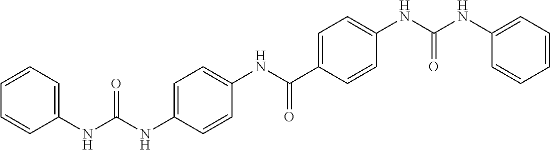 | 79 ± 38 | >240 |
| 5799 | 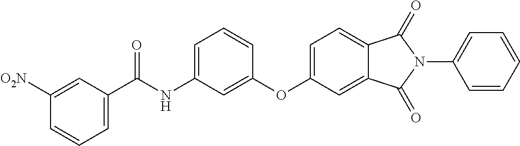 | 202 ± 38 | >240 |
| 5802 | 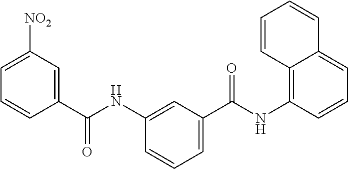 | 225 ± 36 | >240 |
| 5806 | 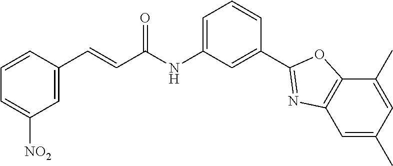 | 68 ± 5 | >240 |
| 5807 | 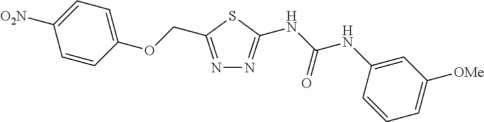 | 124 ± 5 | >240 |
| 5815 | 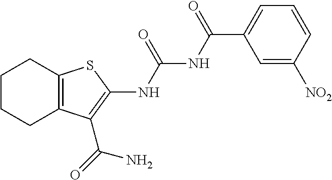 | 186 ± 82 | >240 |
| 5821 | 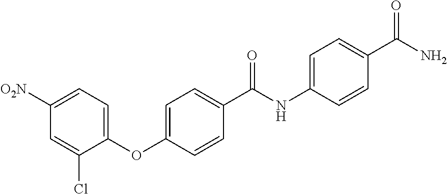 | 130 ± 37 | >240 |
| 5822 | 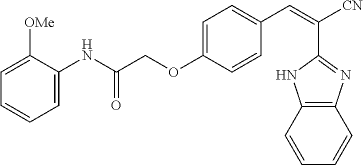 | 178 ± 102 | >240 |
| 5830 | 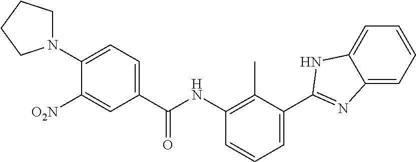 | 193 ± 8 | >240 |
| 5831 | 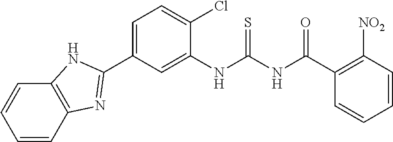 | 243 ± 38 | >240 |

The virtual screening described in Example 1 has provided drug-like small molecule inhibitors of NAD synthetase with antibacterial activity.

### Example 1B

### Virtual Screening to Identify Lead Inhibitors for Bacterial NAD Synthetase (NADs)

The in silico screening program FlexX 2.2.1 (BioSolveIT GMBH; Cologne Area, Germany) was used for the virtual screening of commercially available compounds within the catalytic site of NADs to identify classes of lead inhibitors. The 2008 version of the ZINC drug-like commercial database (˜2.5 million compounds) was docked into the known crystal structure of B. anthracis NADs (McDonald et al. Acta Crystallographica, Section D-Biological Crystallography 2007, 63, 891).

NADs is a large homodimer of approximately 60 kDa that contains two identical binding sites, one within each monomer. The crystal structure (PDB code 2PZ8) of the protein from B. anthracis reveals two identical long, linear binding sites containing the adenylated reaction intermediates lying partly within the dimer interface on the NaAD end, and in a buried cavity within one monomer on the ATP end. In order to generate a less complex crystal structure to utilize in the docking studies, one of the binding sites was isolated by creating a sphere with radius 25 Å around one of the bound intermediates, producing a partial protein structure which consisted of the three shells of amino acid residues immediately surrounding the binding cavity and which fully contained one complete binding site. All crystallographic waters and metals were removed, hydrogens were added, and the protonation states of active site residues were adjusted to their dominant ionic forms assuming a local physiological pH. The “active site,” as needed for use by FlexX, was further defined by creating a smaller sphere of radius 17 Å which consisted of the first two shells of amino acids surrounding the bound substrate, resulting in a rather large active site: 31 Å in length, and a width ranging from 7 Å on the NaAD end to 16 Å on the ATP end.

The ZINC drug-like database was docked as-is into this generated protein structure employing FlexX 2.2.1 standalone version using default parameters, which has been shown to be suitable for exploring many kinds of binding sites (Lyne, P. D. et al., J. Med. Chem. 2004, 47, 1962; Stahl, M. and Rarey, M. J. Med. Chem. 2001, 44, 1035; Luksch, T. et al. Chem. Med. Chem. 2008, 3, 1323) and routinely produces hit rates comparable to other highly regarded programs (Kontoyianni et al. J. Comput. Chem. 2005, 26, 11; Bursulaya et al. J. Comput.-Aided Mol. Des. 2003, 17, 755; Rarey et al. Bioinformatics 1999, 15, 243). Automatic base fragment selection was employed, formal charges were assigned to each ligand, and the core subpocket was defined as all residues which interact directly with the bound substrate. Docking was completed in parallel using a 64 bit PQS 16-processor Opteron Quantum Cube running Linux (Advanced Micro Devices, Inc.; Sunnyvale, Calif.). After all ligands were docked and ranked according to FlexX score, minimized structures of the top-scoring 2000 compounds were re-ranked using a consensus scoring program, CScore (Tripos; St. Louis, Mo.) (Yang et al. J. Chem. Inf. Model. 2005, 45, 1134; Wang, et al. J. Chem. Inf. Comput. Sci. 2001, 41, 1422; Dessalew et al. Biophys. Chem. 2007, 128, 165; Forino et al. J. Med. Chem. 2005, 48, 2278). All compounds with a CScore of 5 were reviewed according to several criteria: realistic orientation within the binding pocket, a predicted binding conformation that is energetically reasonable, structures that are chemically simple and can be easily modified synthetically, and compounds representative of chemically diverse structural classes that are considered medicinally interesting. Additionally, selected compounds with both a CScore of 4 and a good FlexX score were reviewed if they were structurally unique. Representatives from the most interesting structural classes were purchased and screened in NADs enzyme inhibition and B. anthracis antibacterial assays.

### Virtual Screening to Identify Lead Inhibitors for Bacterial Nicotinic Acid Mononucleotide Adenylyl Transferase (NaMNAT)

Virtual screening against both an apo site (PDB 3DV2) and a homology model (based on a crystal structure of a B.s. NaMNAT complexed with NaAD (PDB 1KAQ)) of B.a. NaMNAT were carried out using identical procedures, ligands, hardware and software versions as for B.a. NADs described above.

### Example 2

### Materials and Methods

LC/MS Purity Assessment. HPLC analysis was performed using an HP1100 series system with diode array detection coupled with a MICROMASS Platform LCZ mass spectrometer (Waters Corporation; Milford, Mass.). A PHENOMENEX Luna 5 μm, C18, 100 Å, 100×4.60 mm column was used for separations (Phenomenex; Torrance, Calif.). The mobile phase was A: H₂O (0.05% formic acid) and B: acetonitrile (0.05% formic acid). The gradient is listed in Table 4. The flow rate was 0.7 mL/min and diode array detection from 190-600 nm was used for each 10 μL injection. The mass spectrometer was equipped with an electrospray ionization (ESI) probe and was operated in both the ESI(+) and ESI(−) mode. Peak height estimation for each analyte was based on baseline integration of peaks observed by the diode array detector.

TABLE 4

| Time (minutes) | % A | % B |
|---|---|---|
| 0.00 | 80.0 | 20.0 |
| 10.00 | 10.0 | 90.0 |
| 11.00 | 10.0 | 90.0 |
| 11.50 | 80.0 | 20.0 |

NMR Internal Standard Purity Assessment. The compounds were examined for purity via an internal standard NMR purity assessment. The stock NMR solution was created by combining CDCl₃ and MeOH-d₄ in a 1:1 ratio; 10% DMSO-d₆ was added to aid in solubility; and hexamethyldisiloxane (HMDSO; NMR grade, Aldrich; St. Louis, Mo.) was added to yield a final HMDSO concentration of 12 μM. A known amount (between 5 and 10 mg) of compound was dissolved into 0.5 mL of the NMR solvent, and the 1H NMR spectrum was recorded using a 400 MHz Bruker spectrometer. Peaks were integrated and calibrated according to a known peak area (methyl, when available; otherwise, a urea NH). Compound purity was determined by comparing the calculated weight based on HMDSO peak integration to the actual weight measured upon sample preparation.

NAD synthetase HPLC Enzyme Assay. The compounds were tested for activity against NAD synthetase (NADs) using the HPLC assay described in Example 1. Briefly, the assay was carried out in two steps: sample preparation and sample analysis. The preparation of sample plates was performed using a BIOMEK FX liquid handling system (Beckman Coulter; Brea, Calif.). The standard reaction volume was 200 μL. The reaction mixture contained 60 mM HEPPS, pH 8.5, 0.5 mM NH₄Cl, 20 mM KCl, 10 mM MgCl₂, 0.1 mM NaAD, 0.2 mM ATP, 6 μg/mL purified B. anthracia NADs, 2.5% (v/v) DMSO, 0.3% BOG, and inhibitors at various concentrations. Compounds were assayed beginning at 600 μM and at doubling dilutions down to 0.6 μM. The reaction was initiated by adding 0.2 mM ATP, and quenched after 10 minutes by adding 50 μL of 6 M guanidine-HCl. The plates were sealed by aluminum tape, and centrifuged at 2500 rpm for 10 minutes in order to pellet any precipitation that may have been caused by the inhibitors. Plates were stored at 4° C. prior to the HPLC analysis.

The HPLC procedure utilized a GILSON 215 Liquid Handler, two GILSON 306 pumps, and a GILSON 170 diode array detector (Gilson; Middleton, Wis.). A Phenomenex Luna 5 μm, C5, 100 Å, 100×4.60 mm column was used for separations (Phenomenex; Torrance, Calif.). The mobile phase was A: 20 mM NaH₂PO₄ pH 6.90 and B: acetonitrile. The gradient was 100% A from 0-3 minutes, to 5% A/95% B from 3-4 minutes for each 20 μL injection. The flow rate was 1.0 mL/min and diode array detection was from 190-400 nm. Peak height estimation for NAD was based on baseline integration. The % inhibition at each inhibitor concentration was calculated by the difference in peak height of NAD compared to reactions without inhibitor. The IC₅₀ was determined from the plot of NAD peak height vs inhibitor concentration, and is defined as the concentration of inhibitor required to produce NAD peak height at 50% of the uninhibited reaction. In developing this assay, peak areas were also used to calculate the IC₅₀ for selected active compounds, and similar results were obtained. Each compound was tested in duplicate, and the IC₅₀ was reported as the average of duplicate runs.

NaMNAT HPLC Enzyme Assay. This assay monitors the production of NaAD in the enzymatic reaction by separating the reactants and products on an HPLC system. The assay system at pH 7.5 contained 50 mM HEPES, 10 mM MgCl₂, 25 μM nicotinic acid mononucleotide (NaMN), 44 μM ATP, 0.3% BOG, 0.25 μg/ml B.a. NaMNAT, and inhibitors at eleven different concentrations (with 2.5% v/v final DMSO concentration). Under these conditions, the NaMN and ATP concentrations were the same as their Michaelis-Menton constants, which we reported previously (Lu, et al. Bacillus anthracis. Acta Crystallographica, Sect F—Struct. Biol. Cryst. Commun. 2008, 64, 893-898, which is herein incorporated by reference). The enzymatic inhibition assay was carried out in 96-well microtiter plates with a total reaction volume of 200 μL.

In each well, 5 μL of DMSO with variable amount of compounds and 170 μL assay buffer containing everything except ATP were first incubated at room temperature for 10 min. The reaction was then initiated by adding 25 μL, of ATP solution, and allowed to proceed for 10 min. Addition of 50 μL of 6M guanidine-HCl stopped the reaction. The reaction mixture was next separated on a 4.6 mm×100 mm SYNERGI® Polar-RP column (Phenomenex; Torrence, Calif.), using a Shimadzu (Columbia, Md.) liquid chromatography system consisting of two pumps, a temperature controlled autosampler with a 12-plate rack changer, a column oven and a photo diode assay (PDA) detector. Separation of NaAD from the other component was achieved in less than 5 min by isocratic elution using 50 mM sodium phosphate as the running buffer at a flow rate of 1.0 mL/min. The peak area at 260 nm was used to quantify NaAD. Percent inhibition was calculated based on the difference in NaAD production between controls (DMSO only) and samples containing the compounds. The IC₅₀ value was determined by plotting % inhibition vs. compound concentrations and is reported as the average of duplicate runs.

Antibacterial Assay. The compounds were screened against Bacillus anthracis Sterne in an antibacterial assay as described in Example 1. Briefly, B.a. Sterne spores were subcultured from stock cultures into Luria-Bertani (LB) broth and incubated for 2-3 hours at 37° C. in ambient air until the OD₆₀₀ measurement reached 0.5 to 0.6 when the bacteria are in mid-log phase. The cultures were diluted 1:1 into LB Broth with an absorbance at 600 nm measuring 0.25 to 0.3, then were added to plates containing 240 μM samples of the compounds to be tested. Compounds were tested at a final DMSO concentration of 1%. The plates were incubated at 37° C., and absorbance at 600 nm was read at 0 h and every hour for 5 hours. Any compounds which inhibited growth of the vegetative cell (as compared to the control containing only DMSO) were screened in the full MIC determination starting at 240 μM and creating doubling dilutions down to 7.5 μM in quadruplicate wells. A plot of cell density vs. time yields inhibition of growth results, and the MIC is defined as the lowest concentration of compound required to completely inhibit growth (100% inhibition). MIC is reported as the average of the four data points acquired for each compound. Controls for each assay measured sterility, B. anthracis Sterne viability, and MIC₁₀₀ for the clinical antibiotic ciprofloxacin hydrochloride (from MP Biomedicals).

All compounds which showed antibacterial action in the LB assay were then assayed according to the Clinical and Laboratory Standards Institute MIC broth microdilution protocol, which standardizes the number of bacteria used in the inoculum as 5×10⁵ cfu/mL, using cation-adjusted Mueller-Hinton (MH) broth, except that measurements were taken at 5 hours, as opposed to 20 hours.

Synthesis. General: Melting points were determined using a Mel-Temp Electrothermal 1201-D apparatus (Barnstead Thermolyne; Dubuque, Iowa) and are uncorrected. All ¹H and ¹³C NMR spectra were recorded on a Bruker 400 MHz (¹H) spectrometer (Bruker Corporation; Billerica, Mass.) using tetramethylsilane (TMS) as internal standard. Reactions were monitored by TLC (Whatman silica gel, UV254, 25 μm plates, GE Healthcare; Waukesha, Wis.), and flash column chromatography utilized Baker silica gel (40 μm), commercially available from Mallinckrodt Baker, Inc. (Phillipsburg, N.J.) in the solvent system indicated. Anhydrous solvents used for reactions were purchased in SureSeal bottles from Aldrich Chemical Co. (St. Louis, Mo.). Other reagents were purchased from Aldrich Chemical Co., Alfa Aesar (Ward Hill, Mass.) or Acros Organics (Geel, Belgium) and used as received. Parallel reactions were carried out in 10 mL screw-cap vials and were agitated by hand. Parallel work-ups were carried out in 50 mL conical Falcon tubes (BD Biosciences; San Jose, Calif.), were concentrated in 15 mL glass vials using a Savant SpeedVac Plus SC210A (Thermo Scientific; Waltham, Mass.), and, where indicated, were purified by parallel silica gel chromatography (gravity) in 10 mL disposable syringes.

### N-(4-Aminophenyl)-N′-(4-nitrophenyl)urea

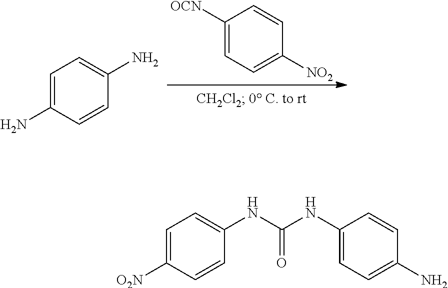

p-Phenylenediamine (12 g, 0.11 mol) was partially dissolved in anhydrous CH₂Cl₂ (60 mL) under a nitrogen atmosphere, and the reaction vessel was submerged in an ice bath. A solution of 4-nitrophenylisocyanate (22 g, 0.13 mol) in anhydrous CH₂Cl₂ (60 mL) was added slowly to the cooled reaction vessel via an addition funnel over a course of 20 minutes with vigorous mechanical stirring, resulting in immediate precipitation of product. Once the addition was complete, the ice bath was removed, and the reaction continued with stirring at room temperature for an additional 20 minutes. TLC (15% i-PrOH in CHCl₃) showed that the diamine and isocyanate starting materials were gone; there was one new product spot (reaction with ninhydrin confirmed the presence of an amine), and one base-line spot corresponding to the diurea byproduct. Solvent was removed under vacuum to obtain a mixture of the two products (crude weight 29 g, 95% yield), which were then stirred in hot acetone (2 L). The diurea byproduct remained insoluble and was filtered off. Solvent was removed under vacuum, and the pure product was obtained as a dense yellow powder (21 g, 71%): mp 221-223° C. (decomposed). ¹H NMR (DMSO-d₆) δ 9.26 (s, 1H, NH), 8.39 (s, 1H, NH), 8.16 (dd, 2H, J=9.33, 3.06 Hz), 7.66 (dd, 2H, J=9.39, 3.06 Hz), 7.09 (dd, 2H, J=8.79, 3.06 Hz), 6.52 (dd, 2H, J=8.76, 3.09 Hz), 4.85 (s, 2H, NH₂). ¹³C NMR (DMSO-d₆) δ 152.18, 146.88, 144.66, 140.59, 127.66, 125.15, 121.18, 117.10, 114.08. MS (ES⁺): m/z 273 (M+H); MS (ES⁻): m/z 271 (M−H).

By this method were also prepared the following:

### N-(4-Aminophenyl)-N′-(3-nitrophenyl)urea

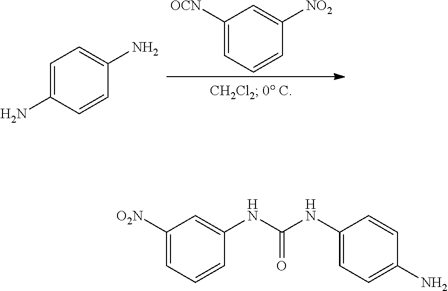

The product was obtained from 3-nitrophenylisocyanate (12 g, 0.11 mol) as a pure yellow powder (8.2 g, 27%): mp 212-214° C. (decomposed). ¹H NMR (DMSO-d₆) δ 9.04 (s, 1H, NH), 8.55 (t, 1H, J=2.21), 8.31 (s, 1H, NH), 7.78 (m, 1H), 7.67, (m, 1H), 7.53 (t, 1H, J=8.15 Hz), 7.09 (dd, 2H, J=8.57, 3.06 Hz), 6.52 (dd, 2H, J=8.56, 3.03 Hz), 4.84 (s, 2H, NH₂). ¹³C NMR (DMSO-d₆) δ 152.76, 148.16, 144.53, 141.55, 129.97, 127.90, 124.01, 121.26, 115.78, 114.08, 111.81. MS (ES⁺): m/z 273 (M+H); MS (ES⁻): m/z 271 (M−H).

### N-(4-Aminophenyl)-N′-(2-nitrophenyl)urea

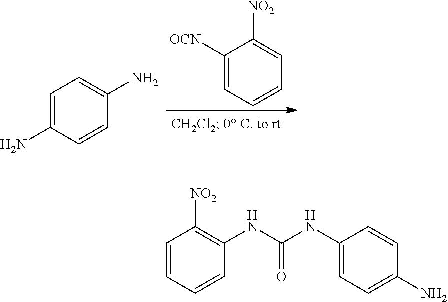

From 2-nitrophenylisocyanate (12 g, 0.11 mol) was obtained the product (14 g, 48%) as a bright orange powder: mp 192-194° C. (decomposed). ¹H NMR (DMSO-d₆) δ 9.51 (s, 1H, NH), 9.36 (s, 1H, NH), 8.34 (d, 1H, J=8.49), 8.08 (dd, 1H, J=8.37, 1.42 Hz), 7.67 (td, 1H, J=7.83, 1.48 Hz), 7.15 (td, 1H, J=7.81, 1.21 Hz), 7.11 (d, 2H, J=8.57 Hz), 6.53 (d, 2H, J=8.55 Hz), 4.88 (s, 2H, NH₂). ¹³C NMR (DMSO-d₆) δ 151.98, 144.75, 136.98, 135.66, 135.04, 127.75, 125.40, 122.14, 121.62, 121.25, 114.11. MS (ES m/z 273 (M+H); MS (ES⁻): m/z 271 (M−H).

### 3,4-Dichloro-(N-(4-(((4 nitrophenyl)amino)carbonyl)aminophenyl))benzene-sulfonamide

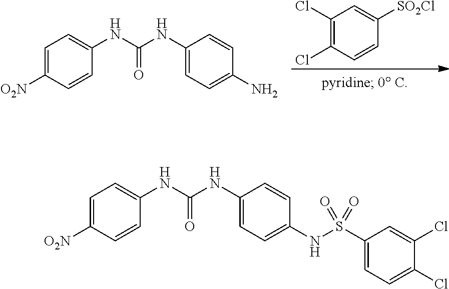

To a solution of N-(4-aminophenyl)-N′-(4-nitrophenyl)urea (1.5 g, 5.5 mmol) in anhydrous pyridine (15 mL) at 0° C. was slowly added 3,4-dichlorobenzenesulfonyl chloride (1.0 mL, 1.6 g, 6.6 mmol). The reaction was stirred under a nitrogen atmosphere for 40 minutes and was diluted with EtOAc (100 mL). The reaction was quenched by adding 2 N HCl (50 mL) and the layers separated; the organic layer was washed further with 2 N HCl (2×50 mL), water (100 mL) and brine (75 mL), and was dried over anhydrous Na₂SO₄. The drying agent was filtered, and the solvent was removed under reduced pressure. The residue (2.1 g, 84%) was taken up in hot methanol (300 mL) and was decolorized with activated charcoal, boiling for 30 minutes. The decolorizing agent was removed by gravity filtration, the filtrate was reduced to 150 mL, and the pure product crystallized to give the product as an off-white solid (1.2 g, 47%): mp 207-209° C. ¹H NMR (DMSO-d₆) δ 10.25 (s, 1H, NH), 9.41 (s, 1H, NH), 8.91 (s, 1H, NH), 8.18 (dd, 2H, J=9.29, 3.03 Hz), 7.89 (d, 1H, J=2.10 Hz), 7.85 (d, 1H, J=8.45 Hz), 7.66 (dd, 2H, J=9.38, 3.11 Hz), 7.63 (dd, 1H, J=8.45, 2.16 Hz), 7.38 (dd, 2H, J=8.96, 2.96 Hz), 7.02 (dd, 2H, J=8.95, 2.97 Hz). ¹³C NMR (DMSO-d₆) δ 151.99, 146.40, 141.09, 139.77, 136.45, 136.04, 132.20, 131.78, 131.36, 128.49, 126.93, 125.25, 122.73, 119.60, 117.56. MS (ES⁻): m/z 479 (M−H).

By this method were prepared the following, with minor changes in purification as noted:

### 3,4-Dichloro-(N-(4-(((3-nitrophenyl)amino)carbonyl)aminophenyl))benzene-sulfonamide

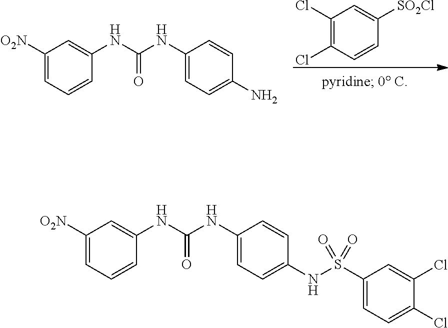

From N-(4-aminophenyl)-N′-(3-nitrophenyl)urea (30 mg, 0.11 mmol) was obtained the product (26 mg, 49%): mp 210.5-212° C. (MeOH). Pure product was obtained by recrystallization from the decolorization solvent MeOH. ¹H NMR (DMSO-d₆) δ 10.22 (s, 1H, NH), 9.18 (s, 1H, NH), 8.81 (s, 1H), 8.53 (s, 1H, NH), 7.88 (s, 1H), 7.82 (m, 2H), 7.65 (m, 2H), 7.54 (t, 1H, J=8.12 Hz), 7.37 (d, 2H, J=8.60), 7.01 (d, 2H, J=8.58 Hz). MS (ES⁻): m/z 479 (M−H).

### 3,4-Dichloro-(N-(4-(((2-nitrophenyl)amino)carbonyl)aminophenyl))benzene-sulfonamide

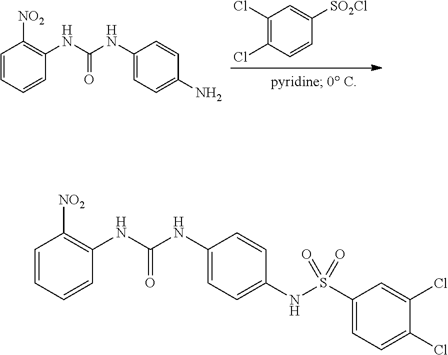

From N-(4-aminophenyl)-N′-(2-nitrophenyl)urea (50 mg, 0.18 mmol) was obtained the product (32 mg, 37%): mp 206.5-208° C. (MeOH). Pure product was obtained by recrystallization from the decolorization solvent MeOH. ¹H NMR (DMSO-d₆) δ 10.22 (s, 1H, NH), 9.83 (s, 1H, NH), 9.56 (s, 1H, NH), 8.26 (d, 1H, J=8.48 Hz), 8.09 (dd, 1H, J=8.32, 1.25 Hz), 7.89 (d, 1H, J=2.13 Hz), 7.85 (d, 1H, J=8.45 Hz), 7.69 (t, 1H, J=7.86 Hz), 7.62 (dd, 1H, J=8.44, 2.12 Hz), 7.39 (d, 2H, J=8.80 Hz), 7.20 (td, 1H, J=7.80, 1.14 Hz), 7.02 (d, 2H, J=8.84 Hz). MS (ES⁻): m/z 479 (M−H).

### Procedure for Parallel Sulfonamide Synthesis:

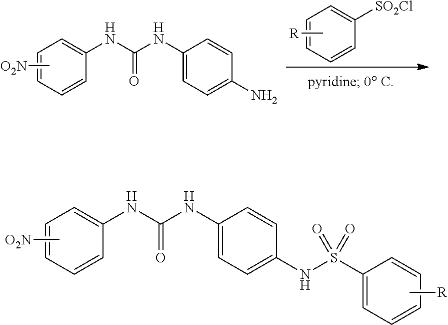

The starting urea-amines (0.55 mmol) were partially dissolved in pyridine (1.5 mL) in 10-mL, screw-cap vials, and the reaction vials were placed in a rack and submerged in an ice bath. The appropriate sulfonyl chlorides (1.2 equiv) were added to each vial; the vials were capped and the entire apparatus was shaken manually at 0° C. for 20 minutes. The vials were removed from the ice bath; reactions were quenched with 1N HCl (1 mL), extracted with EtOAc (3×2 mL), and the organic layers were transferred to 50-mL Falcon tubes. The combined EtOAc extracts were again washed with 1 N HCl (2×2 mL) and water (2×2 mL). Carboxylic acid products were extracted into saturated NaHCO₃ (2×3 mL); the aqueous layers were combined, acidified to pH 3 with concentrated HCl, and extracted with EtOAc (3×5 mL). All products were dried over Na₂SO₄ and the solutions filtered in parallel into 15-mL screw-cap vials. Evaporation of the solvent using the high temperature setting of a speedvac afforded the crude sulfonamide products. All residues were triturated with 6% i-PrOH in CHCl₃ (˜2 mL) to dissolve any unreacted starting materials, and the products were suction filtered to afford the compounds (13-79% yield). LC/MS of these products revealed that most met the 80% purity criteria; those that did not were further purified in parallel by passing through a short silica plug (5×1 cm) using 10-mL syringes and 6% i-PrOH in CHCl₃ as eluent.

### Procedure for Parallel Nitrile Reduction:

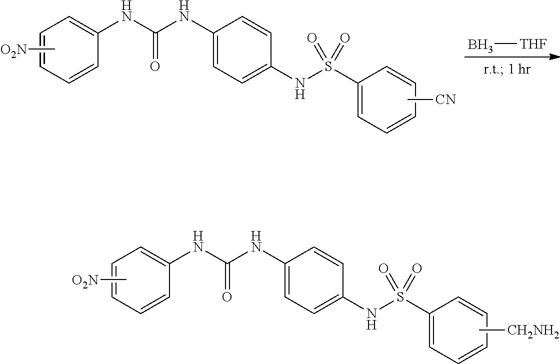

To the starting cyano compounds (i.e., nitriles) (0.080-0.26 mmol) in anhydrous THF (final concentration of V=1.0 M) in 5-mL vials was added BH₃ (1.0 M in THF; 1.3 equiv) at room temperature. The vials were capped, and the reactions stirred under nitrogen for 1 hour. Concentrated HCl was added to quench the excess hydride present in the reaction, and all solvents were removed using the high temperature setting of the speedvac. To the residue was added 2 N NaOH (1 mL), and the amines were extracted into EtOAc (3×2 mL). The organic extracts were combined, washed with water (2 mL) and brine (2 mL), dried over Na₂SO₄, and filtered in parallel into 15-mL vials. Solvent was again removed via the speedvac; residues were taken up in minimal amounts of CHCl₃/i-PrOH (3:1) and purified by silica gel, eluting first with CHCl₃, then gradually increasing polarity to 1:1 CHCl₃/i-PrOH. Column fractions appearing to be at least 80% pure by TLC were combined into 15-mL vials and concentrated to dryness via a speedvac to yield the products (10-63% yield).

### Preparation of Amine Substituted Compounds from Acetamido Substituted Compounds

### 4-Amino-(N-(4-(((2-nitrophenyl)amino)carbonyl)aminophenyl))benzene-sulfonamide

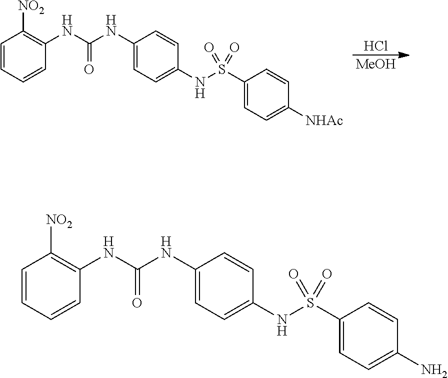

The acetamido product (32 mg, 0.068 mmol) was dissolved in MeOH (1 mL), and concentrated HCl (0.32 mL) was added dropwise. The reaction was stirred overnight at room temperature, and was quenched with 2 N NaOH (0.5 mL). The product was extracted into EtOAc (3×1 mL); the combined extracts were washed with brine (1 mL) and dried over Na₂SO₄. After filtering, the solvent was removed under vacuum, and TLC (15% i-PrOH in CHCl₃) revealed one major new spot. No further purification was pursued, and the product was obtained as an oil (9.4 mg, 32%). MS (ES⁺): m/z 428 (M+H).

### Results

Thirteen compounds exhibited NADs inhibition at or below 300 μM, but did not significantly inhibit bacterial growth. A lack of correlation between NADs inhibition and antibacterial activity was noted. This trend was also observed in previous virtual screening studies, as described in U.S. Provisional Application Ser. No. 61/143,637, which is incorporated herein by reference. Not to be bound by theory, several possibilities may reasonably explain the lack of antibacterial actions for some NADs inhibitors (e.g., may not permeate into the bacterial cell; may be removed by efflux pumps; may undergo metabolism by bacteria). On the other hand, there are several compounds that are antibacterial, but which do not inhibit NAD synthetase, a behavior also exhibited by select compounds in previous studies. These compounds may be inhibiting bacterial growth by some mechanism other than NADs inhibition.

In an attempt to explore the latter, all library members were assayed against the enzyme which immediately precedes NADs in the NAD biosynthetic pathway, nicotinic acid mononucleotide adenylyltransferase (NaMNAT). While NaMNAT contains a smaller catalytic site than NADs, both enzymes share ATP as substrate and bind to an N-ribosylated nicotinic acid. Thus some small molecule inhibitors designed for NADs might reasonably inhibit NaMNAT.

The four most active NaMNAT inhibitors contain R groups that vary from methoxy, to ethyl, to methylamino, to trifluoromethyl, representing four very different substituent types, while the nitrite substituent was not well tolerated. Unlike the NADs inhibition data, a number of different substituents give good NaMNAT inhibition, and there is a relationship between NaMNAT inhibition and antibacterial activity. Twenty antibacterial library compounds had a MH MIC of 30 μM or less. Fifteen of those twenty compounds had a B.a. NaMNAT IC₅₀ of 50 μM or less. Nineteen NaMNAT inhibitors had an IC₅₀ less than 100 μM. Sixteen of these inhibitors also inhibited bacterial growth below 30 μM, although the direct correlation was modest.

Parallel solution-phase synthetic chemistry was utilized to begin exploring the SAR of a new class of drug-like NAD synthetase inhibitors. Seventy-six compounds were synthesized and tested in NADs and NaMNAT enzyme inhibition and B. anthracis antibacterial assays. Though no direct correlation between either NADs or NaMNAT IC₅₀ and MIC was found, all but 3 antibacterial compounds from this compound library inhibited at least one of the enzymes.

### Example 3

### Correlation of Compound Antibacterial Activity within Gram Positive Bacteria

The activity of Compound 5824 was tested against several gram positive bacteria, including B. anthracis, B. cereus, E. faecalis, E. faecium VRE, S. aureus, S. aureus MRSA, and S. pneumoniae. As shown in Table 5, Compound 5824 displays strong antibacterial activity against all gram positive bacteria tested. Further, the data suggests that compounds with strong antibacterial activity against B. anthracis can be predicted to also exhibit strong antibacterial activity against other gram positive bacteria.

TABLE 5
MIC (μg/mL)

| | | | | S. aureus | E. faecium | |
|---|---|---|---|---|---|---|
| B. anthracis | S. aureus | E. faecalis | B. cereus | MRSA | VRE | S. pneumoniae |
| 1.2 | 10.8 | 14 | 0.78 | 5.5 | 10.8 | 3.6 |

### Example 4

### In Vivo Activity of Compounds

Compounds 5824, 5991, 6325, 6333, and 6484 were dissolved and subsequently diluted in a solvent mixture (57.1% PEG 400 (Sigma Aldrich; St. Louis, Mo.), 14.3% ethanol (200 proof) and 28.6% saline) before injection. The stock concentration for each compound was 50 mg/ml. The compounds were tested in vivo for toxicity and pharmacokinetic properties using female BALB/c mice (˜20 g) 6-8 weeks old obtained from Harlan Sprague Dawley, Inc. (Indianapolis, Ind.). Working solutions of the compounds were administered to the mice in groups of three, i.e., three mice for each dosage level, at dosage levels of 0 (control), 10, 25, 50, 100, 250, and 500 mg/kg b.i.d (10 AM and 6 PM) for 3 days. For test Compound 6484, a working solution was administered intraperitoneally at doses of 0 (control), 250, and 500 mg/kg b.i.d for 3 days. A volume of five-fold the body weight (in μL) (0.1 mL/20 g body weight) was injected for the 0 (control), 10, 25, 50, 100, and 250 mg/kg groups; and 10 fold of the body weight (in μL) (0.2 mL/20 g body weight) was injected for the 500 mg/kg groups. The mice were monitored for 7 days after dosing. The toxicities of the compounds were evaluated by determining the maximum tolerated dose (MTD), i.e., the highest dose at which no adverse effects (e.g., piloerection, lowered heads, hunching, and staggering) are observed. The MTD results are shown in Table 6.

TABLE 6

| Compound ID | MTD (mg/kg) |
|---|---|
| 5824 | 25 |
| 5991 | 10 |
| 6325 | 50 |
| 6333 | 250 |
| 6484 | >500 |

Further, pharmacokinetic properties of Compound 5824 were determined by measuring the peak blood levels of the compound. Compound 5824 was dissolved in in 57.1% PEG 400, 14.3% ethanol (200 proof), and 28.6% saline. The final concentrations of the compound were 5 mg/mL and 10 mg/mL (5 mg/mL for 25 mg/kg studies, 10 mg/mL for the 50 mg/kg study).

Female BALB/c mice (20 g, Harlan Sprague Dawley, Inc.) were injected intraperitoneally with 25 mg/kg of Compound 5824 as either a single dose (i.e., QD), with 25 mg/kg twice/day (i.e., Bid), or with a single 50 mg/kg dose. Whole blood was collected in Eppendorf tubes (Eppendorf International; Hamburg, Germany) with heparin at various times [(1) For 25 mg/kg, QD: Predose, 10 min, 30 min, 1 hr, 2 hr, 4 hr, 8 hr, 12 hr, 24 hr and 48 hr; (2) For 25 mg/kg, Bid: Predose, 8 hr, 12 hr, 24 hr and 48 hr (these times are after first dosing, the second dose was given 8 hr after the first); (3) For 50 mg/kg, QD: Predose, 10 min, 30 min, 2 hr, 8 hr, 12 hr and 24 hr.] after administration of the compound, then centrifuged at 14,000 g for 10 minutes to separate plasma. Compound 5824 was extracted from plasma in cold acetonitrile (150 μL plasma was extracted using 300 μL acetonitrile), then dried under a stream of air. Samples were stored at −80° C. until HPLC analysis.

Compound 5824 was safely administered to mice by intraperitoneal injection and was detectable in mouse plasma, after various dosing regimens. The plasma drug concentrations reached the highest concentration after 2 hours in both 25 mg/kg (QD) and 50 mg/kg (QD) groups. For 25 mg/kg (Bid) group, it reached its highest plasma concentration after about 12 hours (4 hours after second dose).

### Example 5

Compounds 6010, 6034, 6399, 6400, and 6572 were evaluated as inhibitors of the human enzymes hNaMNAT-1 and hNaMNAT-3. As shown in Table 7, these compounds displayed low μM inhibition of one or both of these human enzymes. To determine if the hNaMNAT inhibitors have anticancer effects, these compounds were evaluated as in vitro inhibitors of cell growth for 3 different breast cancer cell lines. Several of these compounds proved to be moderate inhibitors of breast cancer cell growth (see Table 7), and the anticancer effects occur selectively at significantly lower concentration than cytotoxicity for normal cells (see Table 8).

Not to be bound by theory, a possible pathway for explaining anticancer effects of human NAD⁺ biosynthesis inhibitors involves poly(ADP-ribose) polymerases (Parp-1 is the most well studied) and the protein deacetylase SirT1 (a member of the sirtuins), two of the most effective NAD⁺-consuming enzymes in the cell. PARP is involved in DNA repair and transcriptional regulation and is now recognized as a key regulator of cell survival and cell death as well as a master component of a number of transcription factors involved in tumor development and inflammation. PARP-1 is essential to the repair of DNA single-strand breaks via the base excision repair pathway, and at least 5 PARP inhibitors are in clinical trials for cancer therapy (Free Radic Biol Med. 2009 Jul. 1; 47(1):13-26). Additionally, inhibition of sirtuins via inhibition of NAD+ availability should also have an anticancer effect. SIRT1 down-regulates the activity of the nuclear transcription factor p53. Thus inhibiting SirT1 would increase p53 activity, thus reducing cancers (Expert Opin Ther Pat. 2009 March; 19(3):283-94).

TABLE 7

| | | | In Vitro Human Breast | | |
|---|---|---|---|---|---|
| | Human NAMNAT | | Cancer | | |
| | Inhibition In Vitro (IC₅₀, μM) | | Inhibition (IC₅₀, μM) | | |
| | hNaMNAT- | hNaMNAT- | | MDA- | |
| Cmpd | 1 | 3 | HCC1937 | MB-436 | SUM159 |
| 6010 | >600 | 12 | 20 | 25 | 10 |
| 6034 | 28 | 7 | 60 | 70 | 50 |
| 6399 | 19 | 24 | 40 | NA | NA |
| 6400 | 13 | 7 | 95 | 95 | 50 |
| 6572 | >600 | >600 | 35 | 35 | 10 |

TABLE 8

| | In Vitro Inhibition Normal Human Cells (IC₅₀, μM) | | | |
|---|---|---|---|---|
| Cmpd | IMR90 | HEK293 | BEAS-23 | Hep-G2 |
| 6010 | >200 | >200 | >200 | >200 |
| 6034 | 148 | 176 | 186 | 90 |
| 6399 | 181 | 163 | 93 | 84 |
| 6400 | >200 | >200 | >200 | 112 |
| 6572 | NA | NA | NA | NA |

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods and combinations of various features of the compounds and methods are intended to fall within the scope of the appended claims, even if not specifically recited. Thus a combination of steps, elements, components, or constituents may be explicitly mentioned herein; however, all other combinations of steps, elements, components, and constituents are included, even though not explicitly stated.

1. A method of treating or preventing a microbial infection or cancer in a subject, inhibiting a bacterial nicotinic acid mononucleotide adenylyltransferase (NaMNAT), a bacterial NAD synthetase, a bacterial NaMNAT and a bacterial NAD synthetase, or inhibiting a human nicotinamide mononucleotide adenylyltransferase (NMNAT), comprising administering to the subject or contacting the bacterial NaMNAT, bacterial NAD synthetase, bacterial NaMNAT, bacterial NAD synthetase, or human NMNAT with an effective amount of a compound of the following structure: 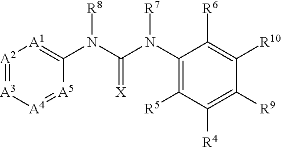
or a pharmaceutically acceptable salt thereof, wherein:
A¹, A², A³, A⁴, and A⁵ are each independently selected from N or CR¹;
R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl;
R⁹ and R¹⁰ are each independently selected from hydrogen and 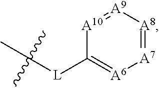
wherein
A⁶, A⁷, A⁸, A⁹, and A¹⁰ are each independently selected from N or CR²; and
L is —SO₂NR³— or —NR³SO₂—,
wherein R⁹ and R¹⁰ are not simultaneously hydrogen; and
X is O or S,
wherein R⁴, R⁵, and R⁶ are hydrogen,
or a composition comprising the compound and a pharmaceutically acceptable carrier.

2. (canceled)

3. (canceled)

4. (canceled)

5. (canceled)

6. (canceled)

7. The method of claim 1, wherein each of A¹, A², A³, A⁴, and A⁵ is CR¹ and each of A⁶, A⁷, A⁸, A⁹, and A¹⁰ is CR².

8. The method of claim 7, wherein one or more of R¹ are each independently selected from hydrogen, nitro, chloro, alkoxyl, or hydroxyl.

9. The method of claim 7, wherein one or more of R² are each independently selected from hydrogen, methyl, ethyl, trifluoromethyl, phenyl, methoxy, phenoxy, amino, methylamino, acetamido, cyano, fluoro, chloro, or carboxyl.

10. (canceled)

11. The method of claim 7, wherein one or more of R² is methylamino, amino, methoxy, ethyl, or trifluoromethyl.

12. (canceled)

13. (canceled)

14. (canceled)

15. (canceled)

16. A compound of the following formula: 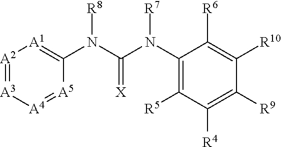
or a pharmaceutically acceptable salt thereof, wherein:
A¹, A², A³, A⁴, and A⁵ are each independently selected from N or CR¹;
R¹, R², R³, R⁴, R⁵, R⁶, R⁷, and R⁸ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl;
R⁹ and R¹⁰ are each independently selected from hydrogen and 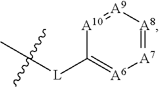
wherein
A⁶, A⁷, A⁸, A⁹, and A¹⁰ are each independently selected from N or CR²; and
L is —SO₂NR³— or —NR³SO₂—,
wherein R⁹ and R¹⁰ are not simultaneously hydrogen; and
X is O or S,
wherein R⁴, R⁵, and R⁶ are hydrogen, and
wherein if A¹, A², A⁴, A⁵, A⁶, and A¹⁰ are each CH, A³ is C—NO₂, R⁴, R⁵, R⁶, R⁷, R⁸, and R¹⁰ are hydrogen, X is O, L is SO₂NH, A⁷ is C—Cl, and A⁹ is hydrogen, then A⁸ is not C—Cl,
if A¹, A², A⁵, A⁷, A⁸, and A⁹ are each CH, A³ and A⁴ are C—Cl, R⁴, R⁵, R⁶, R⁷, R⁸, and R¹⁰ are hydrogen, X is O, and L is SO₂NH, then A⁶ and A¹⁰ are not simultaneously N,
if A¹, A⁴, A⁵, A⁶, A⁷, A⁹, and A¹⁰ are each CH, A² and A³ are C—Cl, R⁴, R⁵, R⁶, R⁷, R⁸, and R¹⁰ are hydrogen, X is O, and L is NHSO₂, then A⁸ is not C—CH₃,
if A¹, A³, A⁴, A⁵, A⁶, A⁸, and A¹⁰ are each CH, R⁴, R⁵, R⁶, R⁷, R⁸, and R¹⁰ are hydrogen, X is O, L is SO₂NH, A⁷ is C—CF₃, and A⁹ is hydrogen, then A² is not C—Cl or CH.

17. A method of treating or preventing a microbial infection or cancer in a subject or inhibiting a bacterial nicotinic acid mononucleotide adenylyltransferase (NaMNAT), a bacterial NAD synthetase, a bacterial NaMNAT, a bacterial NAD synthetase, or a human nicotinamide mononucleotide adenylyltransferase (NMNAT), comprising administering to the subject or contacting the bacterial NaMNAT, bacterial NAD synthetase, bacterial NaMNAT, bacterial NAD synthetase, or human NMNAT with an effective amount a compound of the following structure: 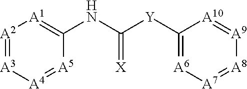
or pharmaceutically acceptable salts or prodrugs thereof, wherein:
A¹, A², A³, A⁴, and A⁵ are each independently selected from N or CR¹;
A⁶, A⁷, A⁸, A⁹, and A¹⁰ are each independently selected from N or CR²,
R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl;
X is O or S; and
Y is —NH—NH—, —NH—CH₂—, an alkyl sulfide, an alkyl carbonyl, or a sulfonamide,
or a composition comprising the compound and a pharmaceutically acceptable carrier.

18. (canceled)

19. (canceled)

20. (canceled)

21. A compound of the following formula: 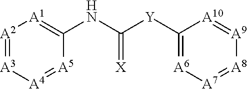
or pharmaceutically acceptable salts or prodrugs thereof, wherein:
A¹, A², A³, A⁴, and A⁵ are each independently selected from N or CR¹;
A⁶, A⁷, A⁸, A⁹, and A¹⁰ are each independently selected from N or CR²,
R¹ and R² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl;
X is O or S; and
Y is —NH—NH—, —NH—CH₂—, an alkyl sulfide, or a sulfonamide,
wherein if A¹ C—OH, A⁵ is CH, A² and A⁴ are CH, A³ is NO₂, A⁶, A⁸, and A¹⁰ are N, X is O, Y is —CH₂—S—, and A⁹ is aniline, then A⁷ is not

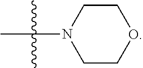

22. A method of treating or preventing a microbial infection or cancer in a subject or inhibiting a bacterial nicotinic acid mononucleotide adenylyltransferase (NaMNAT), a bacterial NAD synthetase, a bacterial NaMNAT, a bacterial NAD synthetase, or a human nicotinamide mononucleotide adenylyltransferase (NMNAT), comprising administering to the subject or contacting the bacterial NaMNAT, bacterial NAD synthetase, bacterial NaMNAT, bacterial NAD synthetase, or human NMNAT with an effective amount a compound of the following structure: 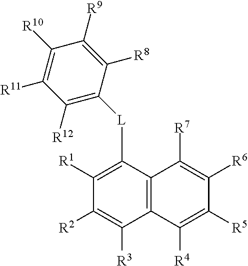
or a pharmaceutically acceptable salt or prodrug thereof, wherein:
L is —SO₂NH— or —NHSO₂—; and
R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl,
or a composition comprising the compound and a pharmaceutically acceptable carrier.

23. The method of claim 22, wherein one of R¹, R², R³, R⁴, R⁵, R⁶, or R⁷ is nitro.

24. (canceled)

25. (canceled)

26. A compound of the following formula: 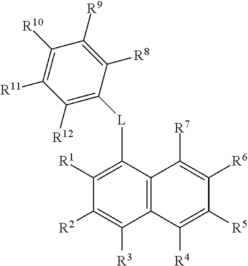
or a pharmaceutically acceptable salt or prodrug thereof, wherein:
L is —SO₂NH— or —NHSO₂—; and
R¹, R², R³, R⁴, R⁵, R⁶, R⁷, R⁸, R⁹, R¹⁰, R¹¹, and R¹² are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, or substituted or unsubstituted carboxyl,
wherein if R¹ is nitro, R², R³, R⁴, R⁵, R⁶, R⁷, R⁸, R⁹, R¹¹, and R¹² are hydrogen, and L is SO₂NH, then R¹⁰ is not ethyl.

27. The method of claim 1, wherein the microbial infection is a bacterial infection.

28. The method of claim 27, wherein the bacterial infection is a gram positive bacterial infection.

29. The method of claim 27, wherein the bacterial infection is a Bacillus anthracis infection.

30. The method of claim 27, further comprising administering a second compound or composition, wherein the second compound or composition includes an antibacterial compound.

31. A composition comprising a compound of claim 16, and a pharmaceutically acceptable carrier.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. The method of claim 1, wherein the cancer is breast cancer.

47. The method of claim 1, further comprising administering a second compound or composition, wherein the second compound or composition includes an anti-cancer agent.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The method of claim 1, wherein the human NMNAT is hNMNAT-1.

61. The method of claim 1, wherein the contacting occurs in vivo or in vitro.

62. (canceled)
